SEQUENCE LISTING (48 sequences)

SEQ ID NO: 1 - protein, 330 aa, Homo sapiens
MEAVAKFDFTASGEDELSFHTGDVLKILSNQEEWFKAELGSQEGYVPKNFIDIQFPKWFHEGLSRHQAENLLMGKEVGFFIIRASQSSPGDFSISVRHEDDVQHFKVMRDNKGNYFLWTEKFPSLNKLVDYYRTNSISRQKQIFLRDRTREDQGHRGNSLDRRSQGGPHLSGAVGEEIRPSMNRKLSDHPPTLPLQQHQHQPQPPQYAPAPQQLQQPPQQRYLQHHHFHQERRGGSLDINDGHCGTGLGSEMNAALMHRRHTDPVQLQAAGRVRWARALYDFEALEDDELGFHSGEVVEVLDSSNPSWWTGRLHNKLGLFPANYVAPMTR

SEQ ID NO: 2 - protein, 322 aa, Mus musculus
MEATAKFDFMASGEDELSFRTGDILKILSNQEEWLKAELGSQEGYVPKNFIDIEFPEWFHEGLSRHQAENLLMGKDIGFFIIRASQSSPGDFSISVRHEDDVQHFKVMRDTKGNYFLWTEKFPSLNKLVDYYRTTSISKQKQVFLRDGTQDQGHRGNSLDRRSQGGPHPSGTVGEEIRPSVNRKLSDHLPLGPQQFHPHQQPSPQFTPGPQPPQQQRYLQHFHQDRRGGSLDINDGHCGLGSEVNATLMHRRHTDPVQLQAAGRVRWARALYDFEALEEDELGFRSGEVVEVLDSSNPSWWTGRLHNKLGLFPANYVAPMMR

SEQ ID NO: 3 - protein, 296 aa, Corvus brachyrhynchos
MEAIAKFDFTASGEDELSFQAGDMLKILSSQEEWYKAELRSHEGYVPKNFIDFHVPPWFDEKISRHEAENLLMSKGVGCFVVRASQNSHGDFSISVRHEDDVQHFKVMRDSKGSYYLWTEKFHSLNKLVDYYKTASISRQKQIFLKDNSQEEKERRGGSLERIGREGFHLGGAAGEDRSAISKRYVEHPIPILQQQQERYGGSLDRKDMLQGLRDHGQGNAGAMQRRHTDPLHQQTPRTLWVRALYDFDAMEHDELGFRSGDILEVLDNSNPSWWKGRLRGELGLFPANYVTPVLL

SEQ ID NO: 4 - protein, 296 aa, Gallus gallus
MEAIAKFDFTASGEDELSFQAGDILKILSSQEEWYKAELRSQEGYVPKNFIDFHVPPWFDEKISRHEAESILMNKGVGSFIVRASQNSHGDFSISVRHEDDVQHFKVMRDSKGNYYLWTEKFYSLNKLVDYYRTSTISRQKQILLRDDSREEKERRGGSLERMSRDGLHVGGAAAEAHSSMSKRYVDHPVPVLHQQQDRYGGSLDRKDALHGLRYHSQGSAAAMQRRHTDPLHQQTPRILWVRALYDFDAVEHDELGFRSGDIVEVLDNSNPSWWKGRLRGELGLFPANYVTPVLL

SEQ ID NO: 5 - protein, 326 aa, Rattus norvegicus
MEAIAKFDFMASGEDELSFHTGDILKILSNQEEWLKAELGSQEGYVPKNFIDIKFPEWFHEGLSRHQAENLLMGKEIGFFVIRASQSSPGDFSISVRHEDDVQHFKVMRDTKGNYFLWTEKFPSLNKLVDYYRTTSISKQKQIFLRDGTQDQQGHRGNSLDRRTQGGPPREVPSGTAGEEIRPLMNRKLSDHLPLGTQQFHPHQQLPPQFPPGPQPPPQQRYLQHFHQDRRGGSLDINDGHLGIGSEMNATLMHRRHTDPVQLQAAGRVRWARALYDFEALEEDELGLRSGEVVEVLDSSNPSWWTGRLHNKLGLFPANYVAPMMR

SEQ ID NO: 6 - protein, 316 aa, Bos taurus
MEAIAKFDFMASGEDELGFHAGDVLKILSNQGEWFKAELGSQEGYVPKNFIEIEFPEWFHEGLSRHQAESLLMGKELGCFIIRASQSSPGDFSISVRHEDDVQHFKVMRDNKGNYFLWTEKFPSLNKLVDYYRKNSISKQKQIFLRDRTREEQGQRGNSLDRRSQGGHPLSGAVGEEIRPSMNRKPSDHPLLPPSQYPPAPLPAQQRYMPQHHFNQERRGASLDINDGPCGLGMSTEMNAALMHRRHTDPVQLQVAGRVRWARALYDFEALEDDELGFRCGEVVEVLDSSNPSWWTGRLHNKLGLFPANYVAPMIR

SEQ ID NO: 7 - protein, 315 aa, Ovis aries
MEAIAKFDFMASGEDELSFHAGDVLKILSNQEEWFKAELGSQEGYVPKNFIDIKFPEWFHEGLSRHQAESLLMGKELGFFIIRASQSSPGDFSISVRHEDDVQHFKVMRDNKGNYFLWTEKFPSLNKLVDYYRKNSISKQKQIFLRDRTREEQGQRGNSLDRRSQGGHPLSGAVGEEMRPSMNRKPSDHPLLPASQYPPAPLQSQQRQHQHHFNQERRGTSLDINDGPCGLGMGTEMNAALMHRRHTDPVQLQVAGRVRWARALYDFEALEDDELGFRCGEVVEVLDSSNPSWWTGRLHNKLGLFPANYVAPMIR

SEQ ID NO: 8 - protein, 318 aa, Capra hircus
MEAIAKFDFMASGEDELSFHAGDVLKILSNQEEWFKAELGSQEGYVPKNFIDIEFPEWFHEGLSRHQAESLLMGKELGFFIIRASQSSPGDFSISVRHEDDVQHFKVMRDNKGNYFLWTEKFPSLNKLVDYYRKNSISKQKQIFLRDRTREEQGQRGNSLDRRSQGGHPLSGAVGEEIRPSMNRKPSDHPLLPASQYPPAPLQSQQRYLPQHQHHFNQERRGTSLDINDGPCGLGMGTEMNAALMHRRHTDPVQLQVAGRVRWARALYDFEALEDDELGFRCGEVVEVLDSSNPSWWTGRLHNKLGLFPANYVAPMIR

SEQ ID NO: 9 - protein, 317 aa, Orcinus orca
MEAIAKFDFMASGEDELSFHAGDVLKILSNQEEWFKAELRSQEGYVPKNFIDIKFPEWFHEGLSRHQAESLLMGKEVGCFIIRASQSSPGDFSISVRHEGDVQHFKVMKDNKGNYFLWTEKFPSLNKLVDYYRTTSISKQKQIFLRDRTREEQGQRGNSLDRRSQGGPPLSGTAGEEIRPSMNRKLSDHPPPPPSQYPPAPLPPQQRYLQQQQPFHQERRGGSLDINDGHCGMGNSSEMNAALMQRRHTDPVQLQAAGRVRWARALYDFEALEDDELGFHCGEVVEVLDSSNPSWWTGRLHNKLGLFPANYVAPMTR

SEQ ID NO: 10 - protein, 316 aa, Equus caballus
MEAIAKFDFTASGEDELSFHTGDVLKILSNQEEWFKAELGSQEGYVPKNFIDIQFPEWFHEGLSRHQAENLLMGKEVGFFIIRASQSSPGDFSISVRHEDDVQHFKVMRDNKGNYFLWTEKFPSLNKLVDYYRTASISRQKQIFLRDRTREDQGRRGSSLDRRLQGGPHLSGAVGEEIRPSMNRKLSDHPPPAPLQYPPAPPPQQQRYLQHPHFHQERRGGSLDINDGHCGMGVGSEMNAALMHRRHTDPVQLQVAGRVRWARALYDFEALEDDELGFRCGEVVEVLDSSNPSWWTGRLHNRLGLFPANYVAPMTR

SEQ ID NO: 11 - protein, 316 aa, Ceratotherium simum simum
MEAIAKFDFTASGEDELSFHTGDVLKILSNQEEWFKAELGSQEGYVPKNFIDIQFPEWFHEGLSRHQAENLLMGKEVGFFIIRASQSSPGDFSISVRHEDDVQHFKVMRDNKGNYFLWTEKFPSLNKLVDYYRTTSISKQKQIFLRDRTREDQGRRGNSLDRRLQGGPHLSGAVGEEIRPSMNRKLSDHPPPAPLQYPPGPPPQQQRYLQHHHLHQERRGGSLDINDGHCGLGMGGEMNAALMHRRHTDPVQLQVAGRVRWARALYDFEALEDDELGFRCGEVVEVLDSSNPSWWTGRLHNKLGLFPANYVAPMTR

SEQ ID NO: 12 - protein, 315 aa, Felis catus
MEAIAKFDFTASGEDELSFHTGDVLKILSNQEEWFKAELGSQEGYVPKNFIDIQFPEWFHEGLSRHQAENLLMGKEVGFFIIRASQSSPGDFSISVRHEDDVQHFKVMRDTKGNYFLWTEKFPSLNKLVDYYRTTSISKQKQIFLRDRTREDQGHRGNSLDRRPQGGPHLSGPGGEEIRPSMNRKLSDHPAPPPSQYPPAPPQQQRYLQHPHFHQERRGTSLDINDGHCGVGMASEMNAALMHRRHTDPVQLQVAGRVRWARALYDFEALEDDELGFRCGEVVEVLDSSNPSWWTGRLHNKLGLFPANYVAPMMR

SEQ ID NO: 13 - protein, 316 aa, Odobenus rosmarus divergens
MEAIAKFDFTASGEDELSFHTGDVLKILSNQEEWFKAELGSQEGYVPKNFIEIQFPEWFHEGLSRHQAENLLMGKEVGFFIIRASQSSPGDFSISVRHEDDVQHFKVMRDNKGNYFLWTEKFPSLNRLVDYYRTTSISKQKQIFLRDRTREDQGHRGNSLDRRPQGGPHLSGAVGEEIRPSMTRKLSDRPAPPPSQCPPAPPAQQHRYLQHPHFHQERRGGSLDINDGHCGMGVGGDMNPALMHRRHTDPVQLQVAGRVRWARALYDFEALEEDELGFRCGEVVEVLDSSNPSWWTGRLHNKLGLFPANYVAPMIR

SEQ ID NO: 14 - protein, 316 aa, Ursus maritimus
MEAIAKFDFTASGEDELSFHTGDVLKILSNQEEWFKAELGSQEGYVPKNFIDIQFPEWFHEGLSRHQAENLLMGKEVGFFIIRASQSSPGDFSISVRHEDDVQHFKVMRDNKGNYFLWTEKFPSLNKLVDYYRTTSISKQKQIFLRDRTREDQGHRGNSLDRRPQGGPHLSGTVGEEIRPSMIRKLSDHPAPPPSPYPPAPPAQQHRYLQHPHFHQERRGGSLDINDGHCGLGVGSEVNAALMHRRHTDPVQLQVAGRVRWARALYDFEALEDDELGFRCGEVVEVLDSSNPSWWTGRLHNKLGLFPANYVAPMIR

SEQ ID NO: 15 - protein, 316 aa, Canis lupus familiaris
MEAIAKFDFTASGEDELSFHTGDVLKILSNQEEWFKAELGSQEGYVPKNFIDIQFPEWFHEGLSRHQAENLLMGKEVGFFIIRASQSSPGDFSISVRHEDDVQHFKVMRDNKGNYFLWTEKFPSLNKLVDYYRTTSISKQKQIFLRDRTREDQGHRGNSLDRRPQGGLFLSGAVGEEIRPPMTRKLSDHPTPPPSQYAPAPPAQQHRYLQHPHFHQERRGGSLDINDGHCGVGMGSEMNAALMHRRHTDPVQLQVAGRVRWARALYDFEALEDDELGFRSGEVVEVLDSSNPSWWTGRLHNKLGLFPANYVAPMIR

SEQ ID NO: 16 - protein, 242 aa, Mus musculus
MEADALSPVGLGLLLLPFLVTLLAALCVRCRELPVSYDSTSTESLYPRSILIKPPQITVPRTPAVSYPLVTSFPPLRQPDLLPIPRSPQPLGGSHRMPSSQQNSDDANSVASYENQEPACKNVDADEDEDDYPNGYLVVLPDSSPAAVPVVSSAPVPSNPDLGDSAFSVESCEDYVNVPESEESAEASLDGSREYVNVSPEQQPVTRAELASVNSQEVEDEGEEEGVDGEEAPDYENLQELN

SEQ ID NO: 17 - protein, 241 aa, Rattus norvegicus
MEADALSPVELGLLLLPFVVMLLAALCVRCRELPASYDSASTESLYPRSILIKPPQITVPRTPATSYPLVTSFPPLRQPDLLPIPRSPQPLGGSHRMPSSRQNSDDANSVASYENQEPARKNVDEDEDEDDYPEGYLVVLPDSSPAAVPVVSSAPVPSNPDLGDSAFSMESCEDYVNVPESEESAEASLDGSREYVNVSQDAQPVIRAELASVTSQEVEDEEEEDVDGEEAPDYENLQELN

SEQ ID NO: 18 - protein, 233 aa, Oryctolagus cuniculus
MEVNVLGHSALGLLLGPLLAVLLMALCVRCRRLSDSYNSASSSDGLYPRSILIKPPYTPAPWPAVTSVPPLSQPDLLPIPRSPQPPGDSHRMPSSRRDSGGAQSVASYENEELACEDADEDEDEEDSHNEGYLEVLPDDSPATSGPGPSAPAPGNPGLRDSAFSMESGDDYVNVPESGGSADASLDGSREYVNVSQELPLEARIQPATLSAPEAGDQEEDEEAPDYENLQGLN

SEQ ID NO: 19 - protein, 261 aa, Felis catus
MISCPRPGSPQLPATGGQALRRSRRLAERQMEAVVLIPYVLGLLLLPLLAVVLCVRCRELPGSYDNTASDSLAPSSIVIKRPPTLATWTPATSYPPVTSYPPLSQPDLLPIPRSPQPPGGSHRMPSSRQDSDGANSVASYENEEPACEDDDEDEEEDYPNEGYLEVLPDSTPATGTAVPPAPAPSNPGLRDSAFSMESGEDYVNVPESEESADVSLDGSREYVNVSQELPPVARTEPAILSSQNDDEEEGAPDYENLQGLN

SEQ ID NO: 20 - protein, 227 aa, Odobenus rosmarus divergens
MEAVVLIPCVLGLLLLPLLAGALCVRCRELPGSYDTTASDSLTPSSIVLKRPPTVVPWPSATSYAPVTSYPSLSQPDLLPIPRSPQPHHVPSSRQDSEGANSVASYENEEPVCEDEEEEDEDYHNEGYLEVLPDSTPAPSSAVPPAPVLSNPGLRDSAFSMESGEDYVNVLESEESADVSLDGSREYVNVSQELPPMAKTEPAILSSQKVEDEDDEEEEGAPDYENL

SEQ ID NO: 21 - protein, 231 aa, Ailuropoda melanoleuca
MEVVVLIPYVLGLLLLPLLAVALCVRCREMPGSYDTGASDSLTPSIVLKRPPTVVPWPSATSYAPVTSYPPLSQPDLLPIPRSPQPHRMPSSRQDSDGANSVASYENEEPVCEDDDEDEEDYHNEGYLEVLPDSTPTTSTAVAPAPVPSNPGLRDSAFSMESGEDYVNVAESEESANVSLDGSREYVNVSQELPPMARTQPAILNSQKLEDEDDEEEEEGAPDYENLQGLH

SEQ ID NO: 22 - protein, 233 aa, Homo sapiens
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 23 - protein, 279 aa, Ovis aries
MEAVSLPVYVLGPLLLPLLAVLLMALCVRCRELPDAESPILHHPLPSSPSFPASRALSSPGSYDTAVSDSLTPSSIMIKLPPTLVPRPSTTPYPLPSQPDLLPIPSPQPLGGSHRMASSRQDSDSANSVASYENEGVTGTPVAPAGRRLRPVLGSADPVSLPPEAACEDADEDDYDEDYNNEGYLVVLPDRDPATSTGNAVSPAPAPSKPGLRDSAFSMESGEDYVNVPESEESADASLNGSREYVNVSQELQPGAKTKPDTQNSQEVEDEEAPDYENL

SEQ ID NO: 24 - protein, 254 aa, Bos taurus
MEAVSPAVYVLGPLLLPLLAVLLMALCVRCRELPGSYDTAVSDSLTPSSIMIKLPPTLVPRPSTTPYPLPSQPDLLPIPRSPQPLGGSHCMASSRQDSASANSVASYENEGVTGTPVAPAGRRLRPILGSADPVWSPPEAACEDADEDDYEEDYNNEGYLVVLPDRDPATSTGNAVSPAPASSKPGLRDSAFSMESGEDYVNVPESEESADASLNGSREYVNVSQELQPGAKTKPDTQNSQEVEDEEAPDYENL

SEQ ID NO: 25 - protein, 254 aa, Capra hircus
MEAVSLPVYVLGPLLLPLLAVLLMALCVRCRELPGSYDTAVSDSLTPSSIMIKLPPTLVPRPSTTPYPLPSQPDLLPIPRSPQPLGGSHRMASSRQDSDSANSVASYENEGVTGTPVAPAGRRLRPVLGSADPVSLPPEAACEDADEDDYDEDYNNEGYLVVLPDRDPATSTGNAVSPAPASSKPGLRDSAFSMESGEDYVNVPESEESAEASLNGSREYVNVSQELQPGAKTKPDTQNSQEVEDEEAPDYENL

SEQ ID NO: 26 - protein, 236 aa, Orycteropus afer afer
MEAAVLVSTVLGLLLMPLLAVLLTVLCARCREMPGSYDSASSDGLYPSNILIKRPYTVAHCPPATSYPPVACHPPLSQPDLLPIPRSPQPPGSSHRIPPSQQDSDGANSVASYENQEPACEDTDGDEDEEDYHNGRYLVVLPDSSPASSTAVPSAPAPSKSGLRDSAFSMESGDEYVNVPESEESAEASLDGSREYVNVSQELQPVARTETATPSSQEVEDEEEGAPDYENLQELN

SEQ ID NO: 27 - protein, 260 aa, Orcinus orca
MEAGSLVLFVLGLLLLPLLAVLLMALCVRCRELPGSYDTAASDGLTPSSIVIKPPPTAASWPPATSYPPVTYLLQSQPDLLRIPRSPQAPGGSPRMPSSQQDSDGANSVASYENEGASGAPAALVGRRLGPVLGSADPVSLPTPEAVCEDADEDEDEEDYPEGYLVVLPDNVPATGAAVPPAPASSNPGLRDSAFSMESGEDYVNVPESEESADASLDGSREYVNVSQELPPVARTEPAGRSSQEVEGEEAPDYENLQIH

SEQ ID NO: 28 - protein, 233 aa, Sus scrofa
MEAVSLIPYALGLLLLPLLAVLLMALCVRCRELPGSYDTAAPDSLTPSSIVIKRPPTLAPWPPATSYPPMTSYPPLSQPDLLPIPRSPQPPGGSHRMPSSRQDSDGANSVASYENEEPVCEDADEDEDEEDYHNEGYLVVLPDSVPATGATVPAAPASNKPGLRDSAFSMESGEDYVNVPESEESADASLNGSREYVNVSQELQPVAKTKPATVRSQEVEDEEAPDYENLQLN

SEQ ID NO: 29 - protein, 268 aa, Ceratotherium simum simum
  misc_feature (261)..(261): Xaa can be any naturally occurring amino acid
MEAAILIPSVLGPLLLPLLAVLLMALCVRCRELPGSYDSAASDSLTPSSIVIKRPPTLAPWPPAASYPPVTSFPPLSQPDLLPIPRSPQPPGGSHRMPSSRQGSDGANSVASYENEGASGTPGAVARGRLRPAPGFADPRVVTPEPACEGADEDEDEEDCHNEGYLVVLPDTVPATGTAIPPPPASKNPGLRDSAFSMESGEDYVNVPESEESAEASLDGSREYVNVSQELQPTARPEPAAWRAREAEDEEEGAPDYENLXELNREPV

SEQ ID NO: 30 - protein, 238 aa, Equus caballus
MEAAILIPSVLGPLLLPLLAVLLMALCVRCRELPGSYDSAATDSLTPSSIVIKRPPTLVPWPPATSYPPVTSFPPLSQPDLLPIPRSPQPPGGSHHMPSSRQGSDGANSVASYENEEPVCEDAEDDEDEEDYHNEGYLVVLPDPAPTTTASTVVPPAPVSKNPGLRDSAFSMESGEDYVNVPESEESADASLDGSREYVNVSQELQPTARTEPATRKAQRVEDEEEGAPDYENLQELN

SEQ ID NO: 31 - protein, 29 aa, Artificial Sequence
  short peptide
  MISC_FEATURE (3)..(3): phosphorylated tyrosine
DDYVNVPESGESAEASLDGSREYVNVSQE

SEQ ID NO: 32 - protein, 29 aa, Artificial Sequence
  short peptide
  MISC_FEATURE (3)..(3): phosphorylated tyrosine
  MISC_FEATURE (23)..(23): phosphorylated tyrosine
DDYVNVPESGESAEASLDGSREYVNVSQE

SEQ ID NO: 33 - protein, 4 aa, Artificial Sequence
  short peptide
PGDF

SEQ ID NO: 34 - protein, 4 aa, Artificial Sequence
  short peptide
MRDT

SEQ ID NO: 35 - protein, 9 aa, Artificial Sequence
  short peptide
ASQSSPGDF

SEQ ID NO: 36 - protein, 5 aa, Artificial Sequence
  short peptide
VMRDT

SEQ ID NO: 37 - protein, 100 aa, Artificial Sequence
  short peptide
QFPKWFHEGLSRHQAENLLMGKEVGFFIIRASQSSPGDFSISVRHEDDVQHFKVMRDNKGNYFLWTEKFPSLNKLVDYYRTNSISRQKQIFLRDRTREDQ

SEQ ID NO: 38 - protein, 4 aa, Artificial Sequence
  short peptide
MRDN

SEQ ID NO: 39 - protein, 9 aa, Artificial Sequence
  short peptide
PGDFGVMRD

SEQ ID NO: 40 - protein, 10 aa, Artificial Sequence
  short peptide
PGDFGGVMRD

SEQ ID NO: 41 - protein, 9 aa, Artificial Sequence
  short peptide
PGDFPVMRD

SEQ ID NO: 42 - protein, 5 aa, Artificial Sequence
  short peptide
VMRDN

SEQ ID NO: 43 - protein, 14 aa, Artificial Sequence
  short peptide
ASQSSPGDFGVMRD

SEQ ID NO: 44 - DNA, 3541 bp, Homo sapiens
gatgatgaaaggagaagggaaaggctgcggatggccttcatttcctgctcctggcatctggggattccgagaatgatcccaggacctagatcctatagattccggcagctcttcctgcaaacacaagcccttgttgtgtgcctgagccttgctctccggcctggagacatccatctgcccactctcctaggaactgctgattaccaggaagcaccatcggctctgctgccctccccctggcagagaggcacaatgaccacattatttctcttccagcttcacgttacagcatggaagctgttgccaagtttgatttcactgcttcaggtgaggatgaactgagctttcacactggagatgttttgaagattttaagtaaccaagaggagtggtttaaggcggagcttgggagccaggaaggatatgtgcccaagaatttcatagacatccagtttcccaaatggtttcacgaaggcctctctcgacaccaggcagagaacttactcatgggcaaggaggttggcttcttcatcatccgggccagccagagctccccaggggacttctccatctctgtcaggcatgaggatgacgttcaacacttcaaggtcatgcgagacaacaagggtaattactttctgtggactgagaagtttccatccctaaataagctggtagactactacaggacaaattccatctccagacagaagcagatcttccttagagacagaacccgagaagaccagggtcaccggggcaacagcctggaccggaggtcccagggaggcccacacctcagtggggctgtgggagaagaaatccgaccttcgatgaaccggaagctgtcggatcaccccccgacccttcccctgcagcagcaccagcaccagccacagcctccgcaatatgccccagcgccccagcagctgcagcagcccccacagcagcgatatctgcagcaccaccatttccaccaggaacgccgaggaggcagccttgacataaatgatgggcattgtggcaccggcttgggcagtgaaatgaatgcggccctcatgcatcggagacacacagacccagtgcagctccaggcggcagggcgagtgcggtgggcccgggcgctgtatgactttgaggccctggaggatgacgagctggggttccacagcggggaggtggtggaggtcctggatagctccaacccatcctggtggaccggccgcctgcacaacaagctgggcctcttccctgccaactacgtggcacccatgacccgataaactcttcaggggacagaagctttttgtctggagctgcccacaagaaagagggcaaggaaaaaaggctggactccatgactatatatacatacatctatctacatctgcctgtgtacacacacaactttttatactagtaatttattggcaattgggctggtaattagttgatgcaaaagggaactcaggtggagaataatattgacacttgcttttctgcccccctcaggggtgtgtggaaggcagtgggggagttgggaggggggcagggaaatgaaatggagttttgtcctggccttcagctgtcactgcttcctccttgtcttgggaattttcacggagaacagctaagcagagaccacacctcggcactggacacagaacaacagggtggggttgaacttggtggggcacgttctagctgacctgcaagccccgctcacctggagggcttgcagagcagctctcctcctgttctccaaggggtgggcactgttgcattaggaattaaggtgcagcccagtgctgcgggcagccaagcgagtctgagcggggatgtgaaggcaggacggggttggagagaccctgcctgtgcaaggctggcctcctagtcaacacctagctgagacattcatctctgttcaaattaatattccttgggtctttcaaatgaatgtttcaatgtgaggagcgaagtactgggaaatcgagggattttccacaatccccaagcagcttactcagtggaacctctgtttccccagctatgaaggaaatagcatcctacattccgtaaagtgcttgaagaagctctagctgagaatgctgaattttacagtcattgttttatggttacaagggtatgtcttcagggctctgttcattttccctcttgattttctcttttgaagatgccttgcccagtggcatggaaagaagcccacttgatgataggagcctgactatgaattgattgactaaacatgaccccaggtggaagctggctttgacaccagcttcctgttcagtctgaccactgctgtcctcctgctgcccaagagttgcacctggagcaaggaaccccaccatggctgtcgctctccatcccatcacgctagaatcatgtgtccaagggctcactctggaggtgcacagcacaggtcagcctggccaggggcgaaggagacagtagagaggaagctcagggccttaggggaggccgggtgcaaacccgttctgcaccaagtgcactcggagtttgtgggtatgggtgtgtacccctgcaggtgtgcacatgtgtgcttgcacgcacatatttgtgcactcctgtgtgtatacatgtgtgcttgtgtatgcatatgtgtgcattcctgcatgtgtggacatgtgcgtgcatgcatctgtgtgtctgtgtgtgtgctgagacaggaaagggggtgaaagtgttggtgagggagcctggaagttttctcttccccaacctctcttgctctaaggagggatggggttgggggcagccgttattgaaggtgatcggagaagaaagattttctgactcagaagtgactgccagtgtagcacaagcagtgtcccttgtgactgtgattctacagttctctgatcctcatgtttcctttagaggaaagaggaaaaaaggaactctgtggtgggtattgggagggaaaagaaaatagcctggtggaggcaggagggagtcgagtgtgagtaaggagcacctgcagcttttggaagtgaaagcagagagagggaaaggtagctaagacatccaggaggatcaaggggcagcgtgagaggcacagggggaggaaagggagagggtgagtggggccagggaagagaacggtgtggagttggtgggcagatggtgcaggaggagggctgggcagtggggagaggaagaatcagagcagagaaaccgctgctgcggaaggcagggagcaaagaagcataaggaaaagaacaggagaaagaacaaaggcagagaagacctcctcagctattttggtgctaggtaatgtgaaatgctgcagtaaataaaagccataagtaatgtttgattttacagtatttacaaaccatatgctttaaacagcccacaaactccatccagttgtctgatgatgtgggaggaccagctctagttagacggaggtatgtgattcgattctggtggagactttgtgccttgaaaggcttctcaggaaagcagtaataaaacaaagtgtccttgtttggcc

SEQ ID NO: 45 - protein, 110 aa, Artificial Sequence
  short peptide
MHHHHHHGSMQFPKWFHEGLSRHQAENLLMGKEVGFFIIRASQSSPGDFSISVRHEDDVQHFKVMRDNKGNYFLWTEKFPSLNKLVDYYRTNSISRQKQIFLRDRTREDQ

SEQ ID NO: 46 - protein, 29 aa, Artificial Sequence
  short peptide
DDYVNVPESGESAEASLDGSREYVNVSQE

SEQ ID NO: 47 - DNA, 1554 bp, Artificial Sequence
  Coding sequence (ORF) of the Gads “bait”, encoding N-terminally Ras-tagged full length Human Gads
atgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggcctggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtggatcctggaagctgttgccaagtttgatttcactgcttcaggtgaggatgaactgagctttcacactggagatgttttgaagattttaagtaaccaagaggagtggtttaaggcggagcttgggagccaggaaggatatgtgcccaagaatttcatagacatccagtttcccaaatggtttcacgaaggcctctctcgacaccaggcagagaacttactcatgggcaaggaggttggcttcttcatcatccgggccagccagagctccccaggggacttctccatctctgtcaggcatgaggatgacgttcaacacttcaaggtcatgcgagacaacaagggtaattactttctgtggactgagaagtttccatccctaaataagctggtagactactacaggacaaattccatctccagacagaagcagatcttccttagagacagaacccgagaagaccagggtcaccggggcaacagcctggaccggaggtcccagggaggcccacacctcagtggggctgtgggagaagaaatccgaccttcgatgaaccggaagctgtcggatcaccccccgacccttcccctgcagcagcaccagcaccagccacagcctccgcaatatgccccagcgccccagcagctgcagcagcccccacagcagcgatatctgcagcaccaccatttccaccaggaacgccgaggaggcagccttgacataaatgatgggcattgtggcaccggcttgggcagtgaaatgaatgcggccctcatgcatcggagacacacagacccagtgcagctccaggcggcagggcgagtgcggtgggcccgggcgctgtatgactttgaagccctggaggatgacgagctggggttccacagcggggaggtggtggaggtcctggatagctccaacccatcctggtggaccggccgcctgcacaacaagctgggcctcttccctgccaactacgtggcacccatgacccgataa

SEQ ID NO: 48 - DNA, 1059 bp, Artificial Sequence
  Coding sequence (ORF) of the Gads “prey”, encoding N-terminally myristoylated full length human
atggggagtagcaagagcaagcctaaggaccccagccagcgccggtctagagaattccctcggatcctggaagctgttgccaagtttgatttcactgcttcaggtgaggatgaactgagctttcacactggagatgttttgaagattttaagtaaccaagaggagtggtttaaggcggagcttgggagccaggaaggatatgtgcccaagaatttcatagacatccagtttcccaaatggtttcacgaaggcctctctcgacaccaggcagagaacttactcatgggcaaggaggttggcttcttcatcatccgggccagccagagctccccaggggacttctccatctctgtcaggcatgaggatgacgttcaacacttcaaggtcatgcgagacaacaagggtaattactttctgtggactgagaagtttccatccctaaataagctggtagactactacaggacaaattccatctccagacagaagcagatcttccttagagacagaacccgagaagaccagggtcaccggggcaacagcctggaccggaggtcccagggaggcccacacctcagtggggctgtgggagaagaaatccgaccttcgatgaaccggaagctgtcggatcaccccccgacccttcccctgcagcagcaccagcaccagccacagcctccgcaatatgccccagcgccccagcagctgcagcagcccccacagcagcgatatctgcagcaccaccatttccaccaggaacgccgaggaggcagccttgacataaatgatgggcattgtggcaccggcttgggcagtgaaatgaatgcggccctcatgcatcggagacacacagacccagtgcagctccaggcggcagggcgagtgcggtgggcccgggcgctgtatgactttgaggccctggaggatgacgagctggggttccacagcggggaggtggtggaggtcctggatagctccaacccatcctggtggaccggccgcctgcacaacaagctgggcctcttccctgccaactacgtggcacccatgacccgataa

(19) United States
(12) Patent Application Publication
(10) Pub. No.: US 2020/0172604 A1
(43) Pub. Date: Jun. 4, 2020

(54) AGENTS WHICH INHIBIT GADS DIMERIZATION AND METHODS OF USE THEREOF

(71) Applicant: Technion Research & Development Foundation Limited, Haifa (IL)

(72) Inventor: Deborah YABLONSKI, Zichron Yaakov (IL)

(73) Assignee: Technion Research & Development Foundation Limited, Haifa (IL)

(21) Appl. No.: 16/615,411

(22) PCT Filed: May 22, 2018
(86) PCT No.: PCT/IL2018/050556

§ 371 Date: Nov. 21, 2019

Related U.S. Application Data
(60) Provisional application No. 62/509,795, filed on May 23, 2017.

(51) Int. Cl. C07K 16/18; C07K 14/49; C07K 14/435

(52) U.S. Cl. CPC C07K 16/18; C07K 14/49; A61K 38/00; C07K 14/43509; C07K 2317/76; C07K 14/435

(57) ABSTRACT

Agents which inhibit Gads dimerization are provided. Accordingly there is provided an agent which inhibits Gads (SEQ ID NO: 1) dimerization, the agent interacting with a pharmacophore binding site comprising an amino acid selected from the group consisting of F55, P56, W58, F59, E61, G62, A84-F92, V107-N111, Y115, F116, L125 and N126 of SEQ ID NO: 1. Also provided an agent which inhibits Gads (SEQ ID NO: 1) dimerization, the agent interacting with a pharmacophore binding site comprising an amino acid sequence of an SH3 domain of SEQ ID NO: 1. Also provided are methods of inhibiting activation of a T cell and/or a mast cell and methods of treating or preventing a disease associated with activation of T cells or an allergic response.

### FIELD AND BACKGROUND OF THE INVENTION

The present invention, in some embodiments thereof, relates to agents which inhibit Gads dimerization and methods of use thereof.

The initiation of T cell antigen receptor (TCR) signaling is a key step that can result in T cell activation and the orchestration of an adaptive immune response. Similarly, activation of mast cells, the central mediators of allergic diseases, largely depends on activation through the specific receptor for IgE (FcεRI): cross-linking of FcεRI on mast cells initiates a cascade of signaling events that eventually results in degranulation, cytokine/chemokine production and leukotriene release, contributing to allergic symptomology (1).

Upon antigen recognition, the TCR and FcεRI trigger ITAM-dependent signaling cascades, initiated by Src- and Syk-family tyrosine kinases. The Syk-family kinase directly phosphorylates two key adaptor proteins: LAT, a membrane-bound adaptor; and SLP-76, a cytoplasmic adaptor (2). LAT is phosphorylated at multiple tyrosine residues, triggering SH2-mediated assembly of large LAT-nucleated signaling complexes (4).

Among the proteins recruited to LAT are the Grb2-family adaptors: Grb2, Grap and Gads (9, 10). Grb2-family adaptors are composed of a central SH2 domain flanked by two SH3 domains, as well as a unique proline rich linker found only in Gads. Located in the cytoplasm, Grb2-family adaptors bind to key signaling proteins via their SH3 domains: Grb2 binds constitutively to SOS, whereas Gads C-terminal SH3 binds with high affinity to an RXXK motif in SLP-76 (13). The central SH2 domain of Grb2-family proteins is specific for phospho-YxN motifs, at least three of which are found in LAT. In this way, Grb2 recruits SOS to LAT, whereas Gads recruits SLP-76 to LAT.

Phospholipase-Cγ1 (PLC-γ1) binds directly to phospho-LAT, and is phosphorylated and activated by a SLP-76-associated tyrosine kinase, ITK, via a multi-step mechanism that depends on the association of ITK with SLP-76 (14,16,17). Gads facilitates PLC-γ1 phosphorylation, by bridging the binding of SLP-76 to LAT (21). Activated PLC-γ1 generates inositol 3 phosphate (IP₃), which triggers elevated intracellular calcium that is required for subsequent transcriptional changes.

The heterotrimeric complex of the adaptors, LAT-Gads and SLP-76, is required for FcεRI-mediated activation of mast cells (22-24), and for TCR-induced activation of T cells (21, 26-34).

At least four tyrosine phosphorylation sites on LAT are required for TCR- or FcεRI-induced PLC-γ1 activation: Y132, 171, 191 and 226 on human LAT (8), or their equivalents in mouse LAT (53). PLC-γ1 binds selectively to pY132, whereas Gads and Grb2, by virtue of their similar SH2 domains, bind to pYxN motifs at tyrosines Y171, Y191 and Y226 (25), suggesting that they may compete for binding sites on LAT.

Both Y171 and Y191 are required for stable binding of Gads to LAT and for downstream responsiveness, suggesting the possibility of cooperative binding to LAT (8). Previous reports have presented evidence that Gads undergoes oligomerization, which may profoundly influence its binding to phospho-LAT and consequently cell responsiveness to stimulation (FASEB conference on Signal Transduction in the Immune system, Jun. 7-12, 2015, Big Sky, Mont., USA, poster and abstract No. 12: “Bridging the Gap: Unexpectedly complex regulation of Gads fine-tunes signaling through the TCR signalosome” by Yablonski, D., Waknin-Lellouche, C., Lugassy, J., Halloumi, E., and Sukenik, S; and EMBO conference on Lymphocyte Antigen Receptor Signaling, Sep. 3-7, 2016, Pontignano (Siena), Italy, poster and abstract No. 99: “Gads dimerization regulates T cell receptor responsiveness”, by Yablonski, D., Sukenik, S., Waknin-Lellouche, C., Halloumi, E., Shalah, R., and Avidan, R.).

### SUMMARY OF THE INVENTION

According to an aspect of some embodiments of the present invention there is provided an agent which inhibits Gads (SEQ ID NO: 1) dimerization, the agent interacting with a pharmacophore binding site comprising an amino acid selected from the group consisting of F55, P56, W58, F59, E61, G62, A84-F92, V107-N111, Y115, F116, L125 and N126 of SEQ ID NO: 1.

According to an aspect of some embodiments of the present invention there is provided an agent which inhibits Gads (SEQ ID NO: 1) dimerization, the agent interacting with a pharmacophore binding site comprising an amino acid sequence of an SH3 domain of SEQ ID NO: 1.

According to some embodiments of the invention, there is provided a pharmaceutical composition comprising, as an active ingredient, the agent of the present invention; and a pharmaceutically acceptable carrier or excipient.

According to some embodiments of the invention, there is provided a method of inhibiting activation of a T cell and/or a mast cell, the method comprising contacting the T cell and/or the mast cell with the agent of the present invention or the pharmaceutical composition of the present invention, thereby inhibiting activation of the T cell and/or the mast cell.

According to some embodiments of the invention, the mast cell activation is FcεRI dependent.

According to some embodiments of the invention, the activation of the mast cell results in at least one of:

(i) calcium flux;

(ii) degranulation; and

(iii) cytokine production and/or secretion.

According to some embodiments of the invention, the T cell activation is TCR dependent.

According to some embodiments of the invention, the T cell is an effector T cell.

According to some embodiments of the invention, the T cell is a regulatory T cell.

According to some embodiments of the invention, the activation of said T cell results in at least one of:

(i) expression of activation markers; and

(ii) phosphorylation of PLC-γ1.

According to some embodiments of the invention, there is provided a method of treating or preventing an allergic response in a subject in need thereof, the method comprising administering to the subject a therapeutically effective amount of the agent of the present invention or the pharmaceutical composition of the present invention, thereby treating or preventing the allergic response in the subject.

According to some embodiments of the invention, there is provided the agent of the present invention or the pharmaceutical composition of the present invention, for use in the treatment or prevention of an allergic response.

According to some embodiments of the invention, there is provided a method of treating or preventing a disease associated with activation of T cells in a subject in need thereof, the method comprising administering to the subject a therapeutically effective amount of the agent of the present invention or the pharmaceutical composition of the present invention, thereby treating or preventing the disease associated with activation of T cells in the subject.

According to some embodiments of the invention, there is provided the agent of the present invention or the pharmaceutical composition of the present invention, for use in the treatment or prevention of a disease associated with activation of T cells.

According to some embodiments of the invention, the T cells are effector T cells.

According to some embodiments of the invention, the disease is an autoimmune disease.

According to some embodiments of the invention, the T cells are regulatory T cells.

According to some embodiments of the invention, the disease is chronic inflammation or cancer.

According to an aspect of some embodiments of the present invention there is provided a method of identifying an agent that inhibits Gads dimerization, the method comprising:

(a) designing a test agent which inhibits Gads (SEQ ID NO: 1) dimerization by interacting with a pharmacophore binding site comprising an amino acid selected from the group consisting of F55, P56, W58, F59, E61, G62, A84-F92, V107-N111, Y115, F116, L125 and N126 of SEQ ID NO: 1; and optionally

(b) testing an effect of the agent on Gads dimerization or a biological outcome thereof.

According to an aspect of some embodiments of the present invention there is provided a method of identifying an agent that inhibits Gads dimerization, the method comprising:

(a) designing a test agent which inhibits Gads (SEQ ID NO: 1) dimerization by interacting with a pharmacophore binding site comprising an amino acid sequence of an SH3 domain of SEQ ID NO: 1; and optionally

(b) testing an effect of the agent on Gads dimerization or a biological outcome thereof.

According to some embodiments of the invention, the agent is a peptide.

According to some embodiments of the invention, the peptide comprises an amino acid sequence selected from the group consisting of PGDF (SEQ ID NO: 33), MRDT (SEQ ID NO: 34), MRDN (SEQ ID NO: 38), PGDFGVMRD (SEQ ID NO: 39), PGDFGGVMRD (SEQ ID NO: 40), PGDFPVMRD (SEQ ID NO: 41), ASQSSPGDF (SEQ ID NO: 35), VMRDT (SEQ ID NO: 36), VMRDN (SEQ ID NO: 42) and ASQSSPGDFGVMRD (SEQ ID NO: 43).

According to some embodiments of the invention, the agent is a small molecule.

According to some embodiments of the invention, the agent is an antibody.

According to some embodiments of the invention, the amino acid is selected from the group consisting of A84-F92 and V107-N111.

According to some embodiments of the invention, the SH3 domain is located N-terminally to a SH2 domain of said SEQ ID NO: 1.

According to some embodiments of the invention, the SH3 domain comprises an amino acid sequence of SEQ ID NO: 50.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-J demonstrate spontaneous Gads dimerization which is mediated by its SH2 domain, and stabilized by the N-terminal SH3. FIG. 1A is a histogram demonstrating full length MBP-tagged Gads (MBP-Gads) as resolved by size exclusion chromatography on a Superdex 200 10/300 GL column. FIG. 1B is a graph demonstrating thermal stability of purified MBP-Gads protein as determined by nano-DSF. Triplicate samples of MBP-Gads from peaks 1 (monomer, shades of green) and 2 (dimer, shades of purple) were heated at a rate of 1° C./min, while measuring intrinsic tryptophan fluorescence; the resulting Tm is indicated for each peak. FIG. 1C is a schematic representation of constructs encoding wild type (WT) MBP-Gads and MBP-Gads lacking the indicated domain (marked by “X”). FIG. 1D is a histogram demonstrating His-tagged Gads SH2 (His-SH2) as resolved by size exclusion chromatography on a Superdex 75 10/300 GL column. FIG. 1E shows histograms of MBP-Gads protein from the dimeric fraction, either full length (left histogram) or SH2 only (right histogram) as resolved by analytical size exclusion chromatography following incubation at 37° C. for the indicated time. FIG. 1F shows a graph demonstrating thermal stability of His-SH2 as determined by nano-DSF. Triplicate samples of monomeric (peak 1, shades of green) or dimeric (peak 2, shades of purple) His-SH2 were heated at a rate of 1° C./min, while measuring intrinsic tryptophan fluorescence; the resulting parameters for each peak are shown on the right. FIG. 1G demonstrates in-vivo self-association of full length Gads, as determined by the Ras Recruitment System (RRS). The well-characterized interaction of Gads and SLP-76 served as a positive control. FIG. 1H demonstrates the lack of in-vivo self-association of Gads lacking an N-terminal SH3 domain, as determined by the Ras Recruitment System (RRS). FIG. 1I shows representative histograms of MBP-Gads protein from the dimeric fraction, either full length (left histogram) or lacking the N-terminal SH3 (right histogram) as resolved by analytical size exclusion chromatography following incubation at 37° C. for the indicated time. FIG. 1J shows the quantitative analysis of histograms from the experiment shown in FIG. 1I, which were deconvoluted into their constituent dimeric and monomeric components, using the Solve Excel plugin, to determine the fraction of Gads protein, either full length (solid line) or lacking the N-terminal SH3 (dotted line) that remained in the dimeric configuration following incubation at 37° C. for the indicated time.

FIGS. 2A-C demonstrate the molecular weight of monomeric and dimeric forms of Gads. FIG. 2A is a molecular weight calibration curve for the Superdex 200 10/300 GL column obtained by resolving marker proteins from the GE Gel Filtration HMW Calibration Kit (blue). Full length MBP-Gads has a predicted molecular weight of 82 kDa, while MBP-SH2 alone has a predicted molecular weight of 56 kDa. Red and Green symbols mark the relative elution volumes (Vₑ/Vₒ) of the two main peaks observed for the full length MBP-Gads and MBP-SH2 alone, respectively. FIG. 2B shows SDS-PAGE photographs of proteins from the two peaks resolved by size exclusion chromatography of full length of MBP-Gads (as shown in FIG. 1A) (left photograph) and His-Gads SH2 (as shown in FIG. 1D), stained with coomassie blue. FIG. 2C show the molecular weight of MBP-Gads protein from the two peaks, based on SEC-MALS light scattering.

FIG. 3 is a graph demonstrating thermal stability of monomeric and dimeric forms of MBP-Gads SH2. Purified Monomeric or dimeric MBP-Gads SH2 was analyzed by nano-DSF, as in FIG. 1B. Data are from three independent repeats, with monomer shown in shades of green and dimer in shades of purple. In the inset: magnified view of a subtle transition observed in the dimeric form at approximately 33° C.

FIGS. 4A-D demonstrate the identification of the Gads SH2 dimerization interface. FIG. 4A shows Gads dimerization interface. On the left, murine Gads SH2 domain co-crystallized with a short peptide encompassing LAT pY-171 (from PBD file 1R1P, (37)). Shown are two adjacent SH2 units (cyan and green, respectively) bound to a phospho-LAT peptide (red). Dotted box indicates the putative dimerization interface. On the right, enlarged view of part of the dimerization interface, highlighting the position of F92 (shown in space-filling form), D91 and R109. FIGS. 4B and 4C are histograms demonstrating purified MBP-Gads SH2 proteins (FIG. 4B) or full length MBP-Gads proteins (FIG. 4C) bearing the indicated point mutations as resolved by size exclusion chromatography. FIG. 4D shows representative isotherms for the interaction of monomeric MBP-Gads SH2 with pY171-LAT peptide. Data analysis was performed with Affinimeter, using a 1:1 stoichiometry binding model. Shown are the K_{A} and ΔH obtained upon linked-parameter analysis of three repeats for each experiment.

FIGS. 5A-F demonstrate Gads conserved residues which constitute the dimerization interface. FIG. 5A shows a space filling (right picture) and a ribbon (left picture) representation of two adjacent murine Gads SH2 units from PDB file 1R1P. FIGS. 5B-C are a model (FIG. 5B) and a respective Table (FIG. 5C) showing the position of 24 evolutionarily conserved residues found within the dimer interface. 14 core residues are marked in red in FIG. 5C. The human Gads numbering corresponds with Refseq accession number NP_001278754.1 (SEQ ID NO: 1). The mouse Gads numbering corresponds with Refseq accession number NP_034945.1 (SEQ ID NO: 2). FIG. 5D is multiple sequence alignment analysis of Gads SH2 from 15 mammalian and bird species demonstrating position and conservation of the dimerization interface residues. SH2 residues identical in all species are highlighted in yellow. 24 residues from the dimerization interface, as listed in 5C, are highlighted in red on the sequence of human Gads SH2. Sequences included in this alignment were from Corvus brachyrhynchos (XP_008634684.1, SEQ ID NO: 3), Gallus (XP_001234082.2, SEQ ID NO: 4), Rattus norvegicus (NP_001030116.1, SEQ ID NO: 5), Mus musculus (NP_034945.1, SEQ ID NO: 2), Bos Taurus (NP_001179489.1, SEQ ID NO: 6), Ovis aries (XP_012031064.1, SEQ ID NO: 7), Capra hircus (XP_005681147.1, SEQ ID NO:8), Orcinus orca (XP_004279556.1, SEQ ID NO: 9), Homo sapiens (NP_001278754.1, SEQ ID NO: 1), Equus caballus (XP_001502077.1, SEQ ID NO: 10), Ceratotherium simum (XP_004437938.1, SEQ ID NO: 11), Felis catus (XP_003989325.1, SEQ ID NO: 12), Odobenus rosmarus divergens (XP_004397498.1, SEQ ID NO: 13), Ursus maritimus (XP_008703755.1, SEQ ID NO: 14), and Canis lupus (XP_849706.1, SEQ ID NO: 15). FIG. 5E shows a face on view of the dimerization interface looking through the blue subunit towards the yellow. FIG. 5F shows a rotated view of the dimerization interface of the yellow subunit. Note the location of R109 and F92 at the center of the interface flanking two pockets (marked by arrows).

FIG. 6 demonstrates that the R109D, R109A and F92A single mutations are not sufficient to disrupt Gads SH2 dimerization. Shown is a histogram of purified recombinant MBP-Gads SH2 domain, either wild type or bearing the indicated point mutations as resolved by size exclusion chromatography as in FIG. 1A.

FIG. 7 is a graph demonstrating thermal stability of purified monomeric full length MBP-Gads, either wild type (WT, shades of green) or bearing the indicated mutations (shades of blue and red) as determined by nano-DSF performed as in FIG. 1B.

FIGS. 8A-B show multiple sequence alignment analysis of the C-terminal region of LAT, from 15 mammalian species. Identical residues in all species are highlighted in yellow. Four well-characterized phosphoryrosine sites are labeled in red. The Gads-binding sites are found within a highly conserved region, marked by a black box. The sequences used in the alignment were from Mus musculus (NP_034819.1, SEQ ID NO: 16), Rattus norvegicus (NP_110480.1, SEQ ID NO: 17), Oryctolagus cuniculus (XP_008256179.1, SEQ ID NO: 18), Felis catus (XP_003998791.2, SEQ ID NO: 19), Odobenus rosmarus divergens (XP_012416769.1, SEQ ID NO: 20), Ailuropoda melanoleuca (XP_002927386.1, SEQ ID NO: 21), Homo sapiens (AAC39636.1, SEQ ID NO: 22), Ovis aries (XP_011959708.1, SEQ ID NO: 23), Bos taurus (NP_001098448.1, SEQ ID NO: 24), Capra hircus (XP_005697693.1, SEQ ID NO: 25), Orycteropus afer afer (XP_007948358.1, SEQ ID NO: 26), Orcinus orca (XP_004269051.1, SEQ ID NO: 27), Sus scrofa (XP_003124570.1, SEQ ID NO: 28), Ceratotherium simum simum (XP_004439575.1, SEQ ID NO: 29) and Equus caballus (XP_005598878.1, SEQ ID NO: 30). The sequence included in the LAT peptides pY171-LAT (SEQ ID NO: 31) and 2pY-LAT (SEQ ID NO: 32) is outlined in red in FIG. 8A, and shown in FIG. 8B.

FIGS. 9A-D demonstrate preferentially paired binding of the Gads SH2 to its dual sites on LAT. FIG. 9A is a schematic representation of two possible modes of Gads binding to 2pY-LAT. FIGS. 9B-C are histograms demonstrating altered FPLC mobility of Gads upon binding to mono- or dual-phosphorylated LAT peptides. In FIG. 9B 0.7 μM MBP-Gads SH2, from the monomeric (red) or dimeric (blue) fraction was incubated on ice for 15 minutes either alone (solid line) or in the presence of 5 μM pY171-LAT (SEQ ID NO: 31, dashed line) or 2pY-LAT (SEQ ID NO: 32, dotted line); and then resolved by size exclusion chromatography. In FIG. 9C, 0.7 μM full length MBP-Gads from the monomeric fraction, was incubated for 15 minutes at 37° C., either alone (solid line), or in the presence of 5 μM 2pY-LAT peptide (SEQ ID NO: 32, dotted line). FIG. 9D is a histogram demonstrating stabilization of the dimeric form of Gads SH2 upon binding to LAT. 20 μM MBP-Gads SH2 from the dimeric fraction was incubated for 30 minutes on ice (blue) or at 37° C. for 15 minutes (red, solid line), followed by an additional 15 minutes at 37° C. in the presence of 40 μM of 2pY-LAT (SEQ ID NO: 32, red dotted line); and then resolved by size exclusion chromatography.

FIGS. 10A-F demonstrate that the Gads SH2 dimerization interface supports discrimination between mono- and dual-phosphorylated LAT, and that this discrimination is strengthened by the N-terminal SH3. FIG. 10A is a schematic representation of the modes of Gads binding to competing LAT peptides. Paired binding to 2pY-LAT can proceed sequentially (blue arrows) or by capture of transient Gads dimers (black arrows). Positive cooperativity occurs if the second Gads molecule binds with higher affinity than the first; and results in preferentially paired binding. FIGS. 10B, D and E are histograms demonstrating preferentially paired binding of wild-type Gads to dual-phosphorylated LAT, as determined by size exclusion chromatography. In FIG. 10B, 0.7 μM purified monomeric MBP-Gads SH2, either wild-type or F92D, was incubated for 10 minutes at 37° C. with 5 μM 2pY-LAT (SEQ ID NO: 32), in the absence (black) or presence (solid red) of 10 μM pY171-LAT (SEQ ID NO: 31) competitor peptide; and resolved by size exclusion chromatography. The red curve was deconvoluted into its constituent dimeric (dotted red line) and monomeric (dashed red line) components, using the Solve Excel plugin. In FIG. 10C the competitive binding experiments were performed in triplicate, using varied concentrations of competing pY171-LAT (SEQ ID NO: 31); and analyzed as in FIG. 10B. Shown is the percent of Gads protein found in the dimeric fraction. Error bars representing the standard deviation were too small to depict. In FIG. 10D, 2.5 μM purified monomeric full length MBP-Gads, either wild-type (WT), F92D, or F92A/R109A was incubated for 10 min at 37° C., either alone (grey line) or with 25 μM 2pY-LAT (SEQ ID NO: 32), in the absence (black line) or presence (solid red) of 50 μM pY171-LAT (SEQ ID NO: 31) competitor, and resolved by size exclusion chromatography. In FIG. 10E, 5 μM purified monomeric MBP-Gads, either full length (left) or lacking the N-terminal SH2 (Gads ΔN, right) was incubated for 10 minutes at 37° C., either alone (grey line) or with with 10 μM 2pY-LAT (SEQ ID NO: 32), in the absence (black line) or presence (solid red) of 80 μM pY171-LAT (SEQ ID NO: 31) competitor, and resolved by size exclusion chromatography. In FIG. 10F the competitive binding experiment shown in 10E was performed in triplicate, and analyzed as in FIG. 10B. Shown is the percent of Gads protein found in the dimeric fraction. Error bars represent the standard deviation.

FIGS. 11A-C demonstrate that Gads dimerization is required for TCR signaling. Gads-deficient T cells (dG32 cells) were stably infected with GFP or the indicated alleles of twin-strep-tagged Gads-GFP; and FACS sorting was used to isolate cells within a broad (FIG. 11A) or narrow, homogenous range of GFP expression (FIG. 11B-C). FIG. 11A left histogram shows FACS sorting of the isolated cells. FIG. 11A right graph demonstrates CD69 expression in quadruplicate barcoded samples following overnight stimulation with anti-TCR, normalized to PMA-induced expression, within each of the GFP expression ranges shown at left. Error bars indicate the standard deviation. P values are for TCR-stimulated cells, compared to TCR-stimulated vector: *, p<0.05; **; p<0.005, ***, p<0.0005. Color code represents reconstitution with GFP (green), Gads-GFP wild type (blue), F92D (orange) or F92A/R109A (red). FIGS. 11B-C are western blot photographs demonstrating molecular interactions and downstream signaling events mediated by Gads. In FIGS. 11B-C, the indicated cell lines were stimulated for one minute with anti-TCR (C305) or left unstimulated and lysed. In FIG. 11B, wild type Jurkat T cells, or Gads-deficient dG32 cells, stably reconstituted with the indicated allele of twin-strep-tagged Gads-GFP were stimulated for 1 minute with or without anti-TCR (C305) and Gads was purified with streptactin beads. Co-precipitating SLP-76 and pY132-phosphorylated LAT were detected by western blot. The photograph at bottom shows phosphorylation of LAT in total cell lysates. At right: The ratio of pLAT/Gads from 2 (F92A,R109A), 3 (F92D) or 4 (WT) experiments, normalized to TCR-stimulated WT cells from the same experiment. P values are for TCR-stimulated cells, compared to TCR-stimulated WT-reconstituted cells. *, p<0.05; **; p<0.005, ***, p<0.0005. In FIG. 11C, lysates were analyzed by western blotting with the indicated antibodies. Right: The intensity of PLCγ1 phosphorylation from 4 (F92A,R109A and vector), 7 (F92D) or 8 (WT) experiments, normalized to TCR-stimulated WT cells from the same experiment. P values are for TCR-stimulated cells, compared to TCR-stimulated WT-reconstituted cells. *, p<0.05; **; p<0.005, ***, p<0.0005.

FIGS. 12A-C demonstrate that Gads dimerization is required for FcεRI signaling. Fully differentiated BMMCs, derived from wild type (WT), Gads-deficient (KO), or KO bone marrow that had been retrovirally-reconstituted with Gads-GFP (KO+WT or KO+F92D), were barcoded, mixed together, and sensitized with IgE (anti-DNP), followed by stimulation with DNP-HSA at 37° C. Responses were analyzed by FACS, while gating on matched, narrow regions of Gads-GFP expression within each reconstituted population. FIG. 12A demonstrates the intracellular calcium levels. Left: representative histograms of intracellular calcium levels measured ratiometrically, with 0.6 ng/ml DNP-HSA added at the 60 sec time point. Cells found above the baseline during the last 200 sec were defined as responding cells. Right: a graph showing the percent of responding cells, as a function of stimulant concentration. FIG. 12B demonstrates the percentages of CD63⁺ responding cells, indicating cells that have undergone degranulation. Left: BMMCs were unstimulated (filled histogram) or stimulated for 15 minutes with 1.2 ng/ml of DNP-HSA (open histograms: solid line, WT; and dashed line, F92D), fixed and stained with anti-CD63-PE. The indicated gate defines CD63⁺ responding cells. Middle: The percent of CD63⁺ responding cells, as a function of Gads-GFP expression (n=3, error bars indicate the standard deviation, dotted lines indicate the response of WT and KO cells in the same experiment). Right: The percent of CD63⁺ responding cells as a function of stimulant concentration. FIG. 12C demonstrates the percentages of IL-6⁺ responding cells. Left: cells were stimulated for 4.5 hours with 0.6 ng/ml DNP-HSA (black line) or left unstimulated (filled histograms) and IL-6 expression was analyzed by intracellular staining. The indicated gate defines IL-6⁺ responding cells. Right: The percent of IL-6⁺ responding cells, as a function of stimulant concentration.

FIGS. 13A-B demonstrate that FcεRI-induced surface expression of the degranulation marker, CD107a, depends on Gads dimerization interface. IgE-sensitized cells were stimulated for 15 minutes with DNP-HSA, fixed and stained with anti-CD107a-APC. FIG. 13A is a graph of the percent of CD107a⁺ responding cells as a function of Gads-GFP expression (n=3, error bars indicate the standard deviation, DNP-HSA concentration=1.2 ng/ml). FIG. 13B is a graph of the percent of CD107a⁺ responding cells within a single GFP-expression gate, as a function of stimulant concentration.

FIGS. 14A-C demonstrate the principle of designing and/or screening for a Gads dimerization inhibitor, based on two adjacent murine Gads SH2 units from PDB file 1R1P. FIG. 14A is a rotated view of the dimerization interface of the yellow subunit bound to the blue subunit. FIG. 14B is a rotated view of the dimerization interface with selected peptides from the blue subunit, e.g. PGDF (SEQ ID NO: 33) and MRDT (SEQ ID NO: 34). FIG. 14C is a rotated view of the dimerization interface, with core dimerization peptides from the blue subunit encompassing the entire core binding region of the blue subunit to the yellow subunit, i.e. ASQSSPGDF (SEQ ID NO: 35) and VMRDT (SEC ID NO: 36).

### DESCRIPTION OF SPECIFIC EMBODIMENTS OF THE INVENTION

The present invention, in some embodiments thereof, relates to agents which inhibit Gads dimerization and methods of use thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The formation of the LAT-nucleated signaling complex comprising the three adaptors: LAT, Gads and SLP-76 is required for antigen receptor signaling in T and mast cells, via the TCR and FcεRI, respectively. Gads, a Grb2-family adaptor, bridges the TCR and FcεRI-inducible recruitment of SLP-76 to LAT, by binding to LAT through its SH2 domain and binding to SLP-76 through its C-terminal SH3 domain.

Whilst reducing the present invention to practice, the present inventors have now uncovered that dimerization of Gads SH2 domain via its amino acids F55, P56, W58, F59, E61, G62, A84-F92, V107-N111, Y115, F116, L125 and N126 is required for cooperatively paired binding of Gads to adjacent phospho-tyrosine motifs (Y171 and Y191) on LAT; and that Gads signaling functions in both T cells and mast cells depend on this dimerization. In addition, the present inventors have now uncovered that the dimerization of Gads SH2 domain is stabilized by other domains such as the N-terminal SH3 domain.

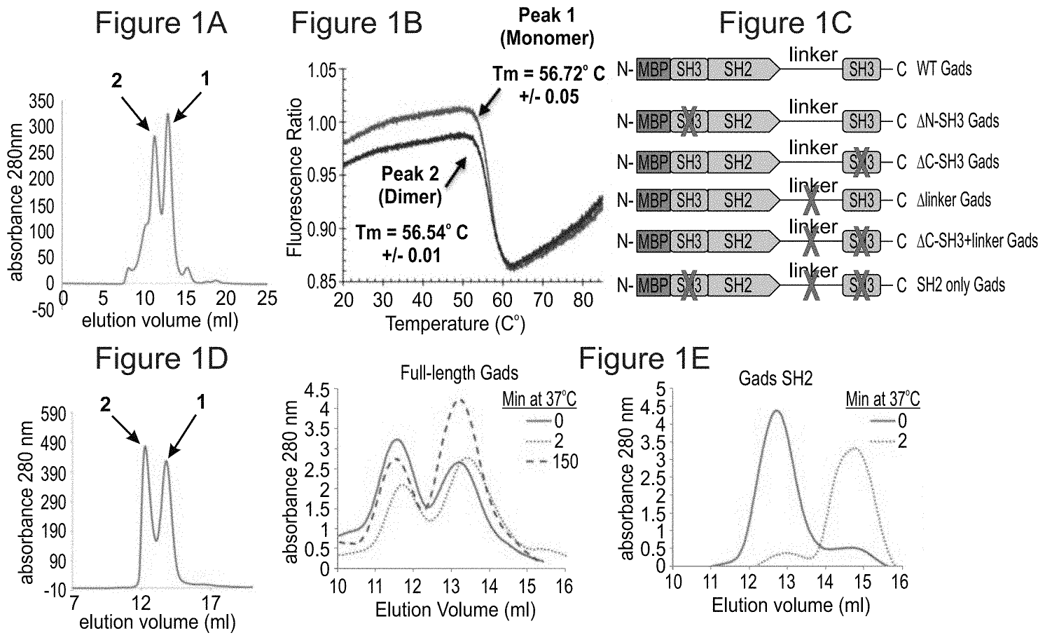

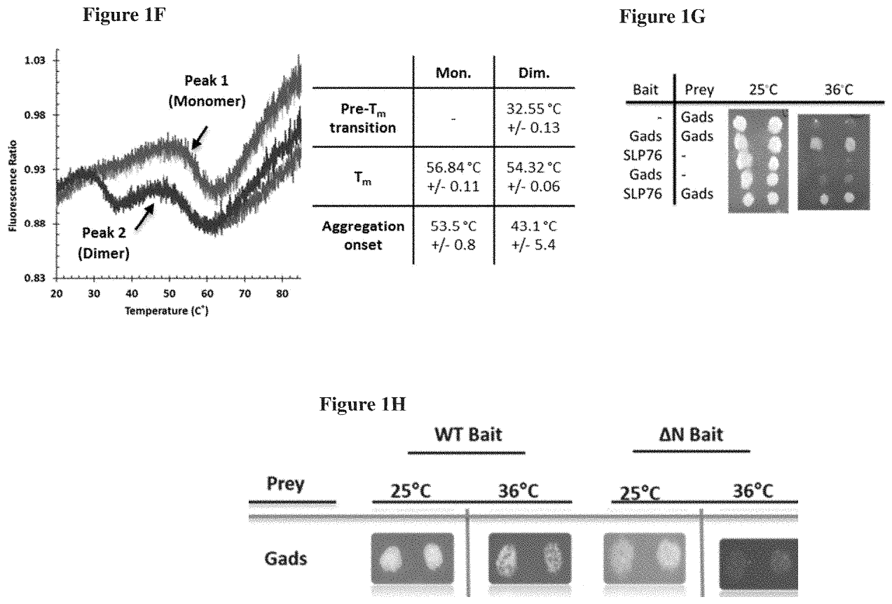

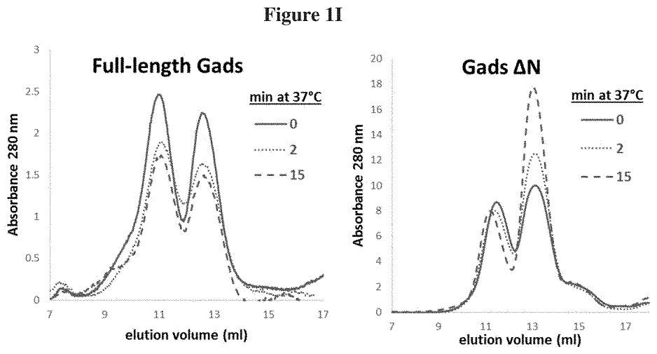

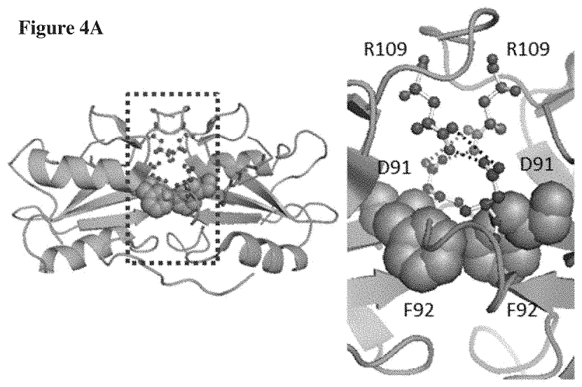

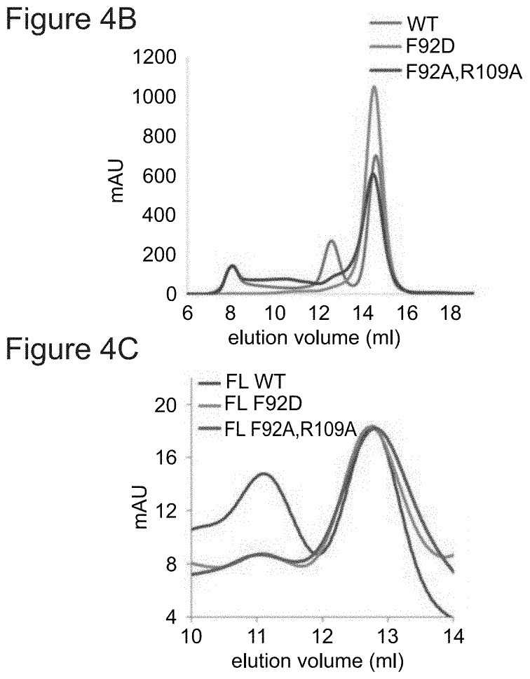

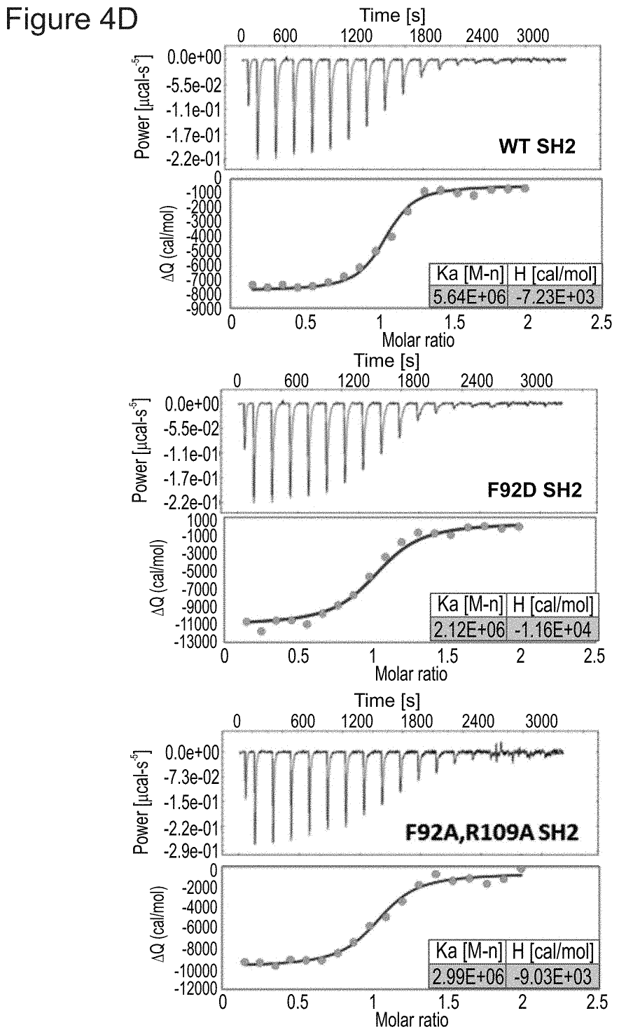

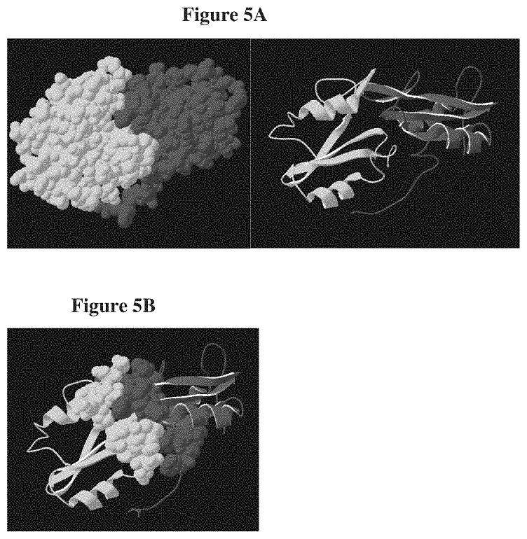

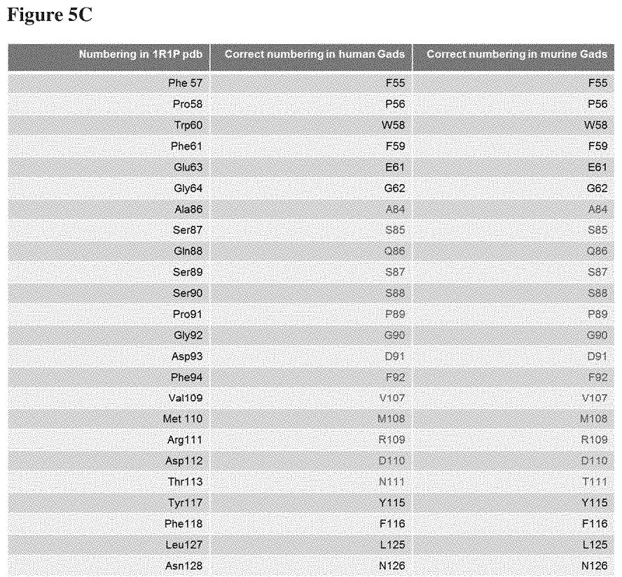

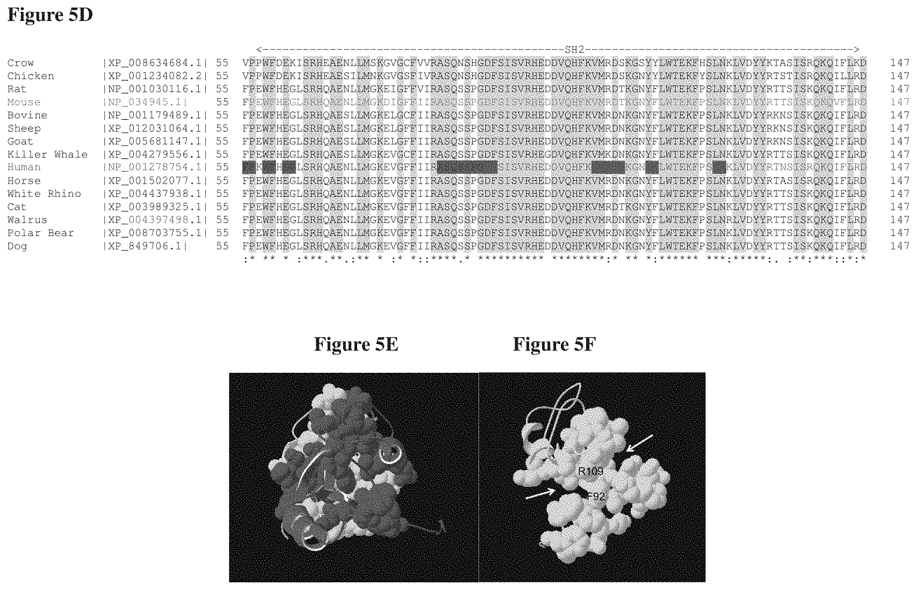

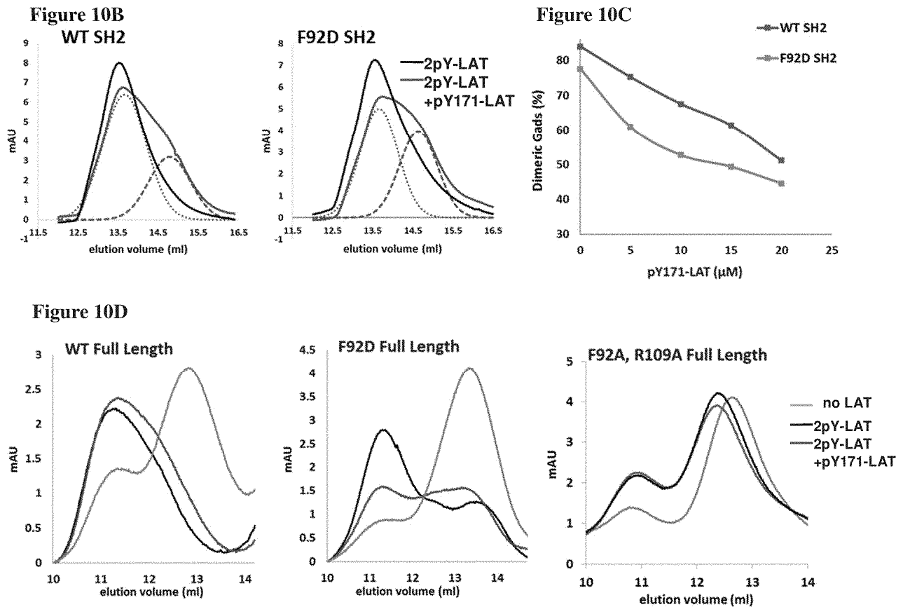

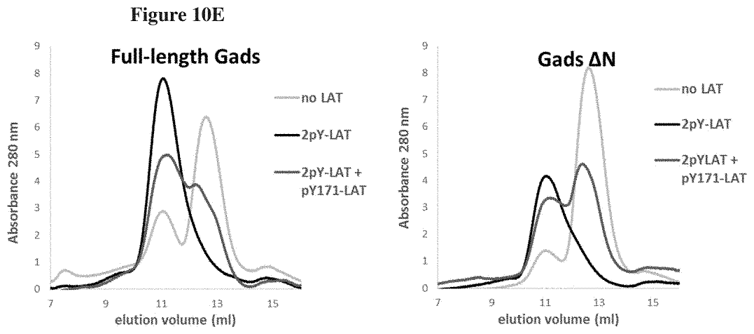

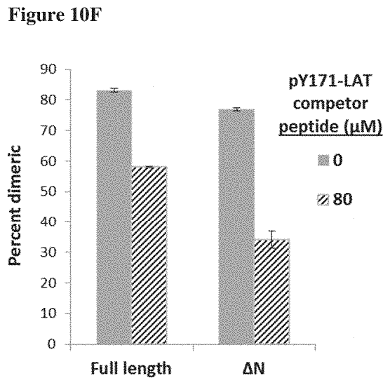

As is illustrated hereinbelow and in the examples section, which follows, the present inventors have discovered spontaneous and LAT-inducible dimerization of Gads and show that dimerization is an intrinsic characteristic of the Gads SH2 domain which discriminates between singly and doubly phosphorylated LAT molecules, by preferentially binding to the latter (Examples 1, 3 and 4 FIGS. 1A-G, 2A-C, 3, 8A-B, 9A-D and 10A-F). The inventors then characterized the dimerization interface based on molecular modeling and identified 24 residues within the dimer interface (namely F55, P56, W58, F59, E61, G62, A84-F92, V107-N111, Y115, F116, L125 and N126), 14 of them were determined to be core residues (Example 2, FIGS. 4A and 5A-D). Furthermore, SH2 domain point mutations designed by the inventors (F92D and F92A/R109A) specifically disrupted Gads dimerization and its cooperative paired binding to doubly phosphorylated LAT, while only moderately affecting the affinity for singly phosphorylated LAT; suggesting that the Gads SH2 dimerization interface is largely distinct from the pTyr-binding pocket (Examples 2-3, FIGS. 4B-D, 6, 7 and 10B-D). Without being bound by theory, the present inventors suggest that transient Gads SH2 dimerization creates an additional binding interface, outside the pTyr-binding pocket, which supports high affinity binding to dual phosphorylated LAT, by interacting with the conserved LAT sequence spanning from pY171 to pY191. In addition, while the SH2 domain was sufficient for dimerization, other domains such as the N-terminal SH3-domain stabilized Gads dimerization at physiologic temperatures and in intact cells (FIGS. 1E-J), and supported the ability of Gads to discriminate between singly- and doubly phosphorylated LAT peptides, by binding preferentially to the latter (FIGS. 10E-F).

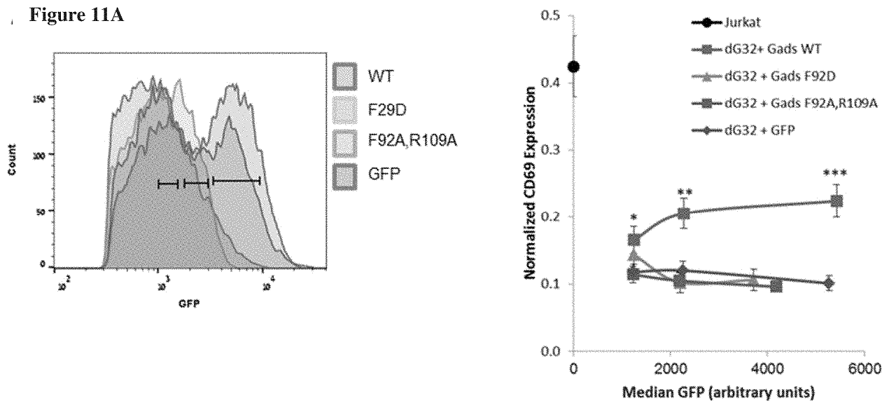

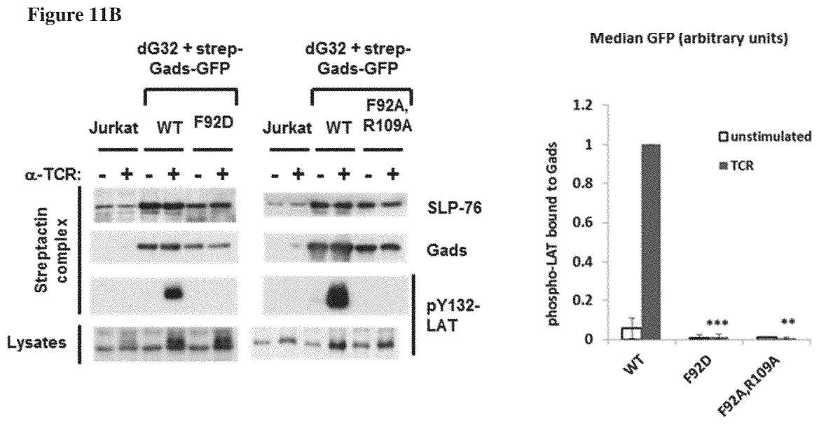

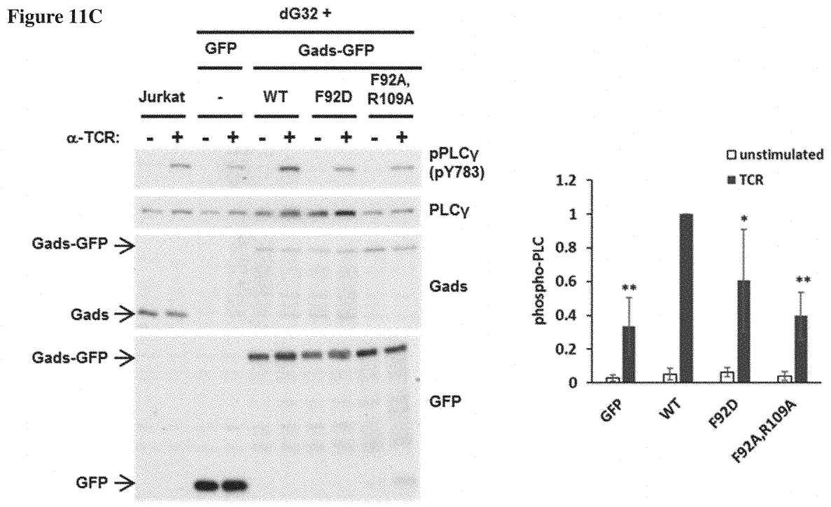

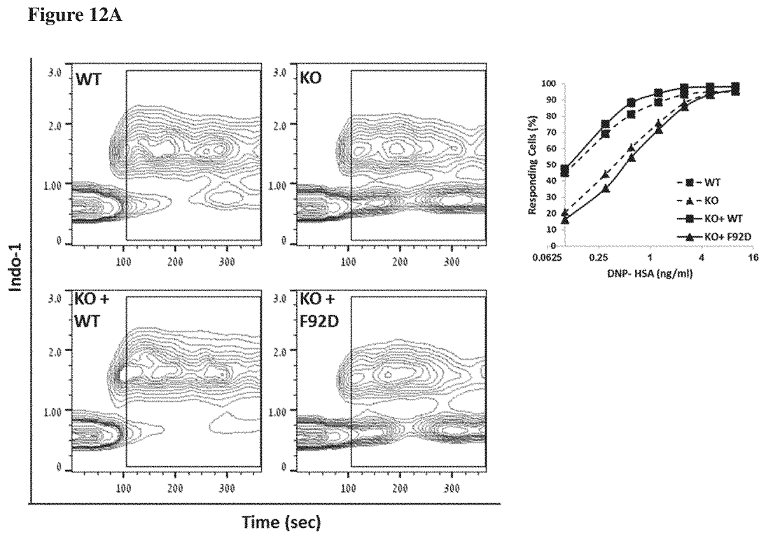

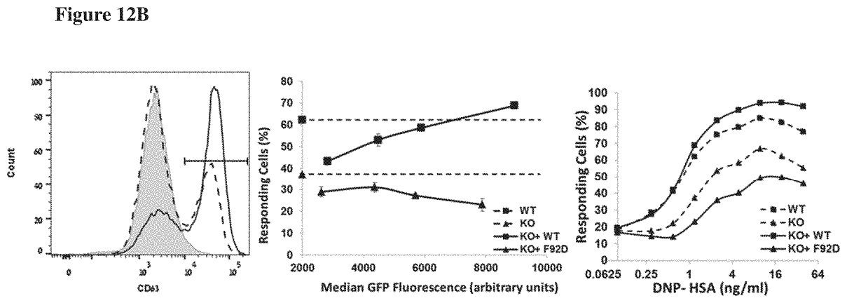

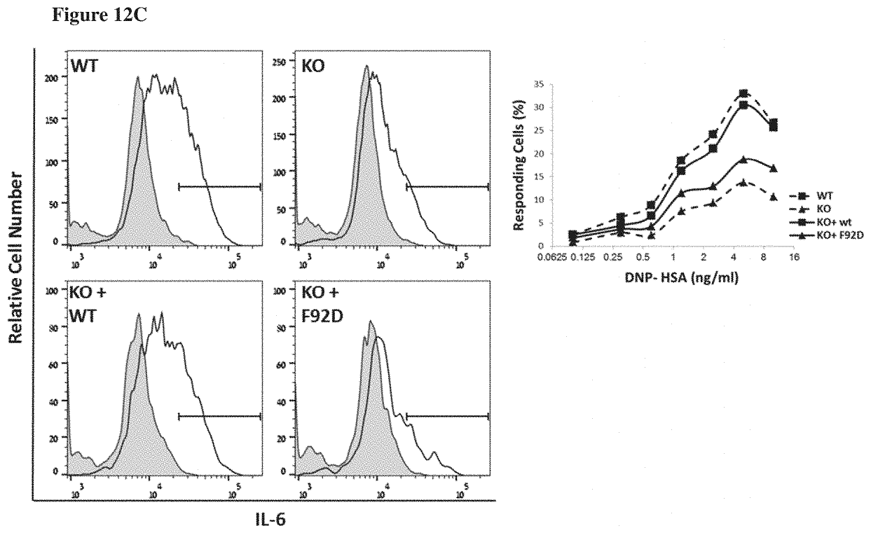

In the next step, the inventors demonstrate that Gads dimerization is required for antigen signaling in T cells and mast cells. Specifically, using a Jurkat-derived Gads-deficient T cell line (dG32), the inventors show that TCR-induced CD69 expression increased following reconstitution with wild type Gads, but not with F92D or F92A/R109A mutated Gads and that mutated Gads abolished TCR-induced recruitment of Gads to phospho-LAT and markedly impaired TCR-induced phosphorylation of PLC-γ1 (Example 4, FIGS. 11A-C); and using Gads-deficient murine bone marrow derived mast cells (BMMCs), the inventors show that following reconstitution with wild type Gads the cells responded to FcεRI antigenic stimulation similarly to wild type BMMCs in terms of calcium flux, degranulation and IL-6 cytokine production, whereas following reconstitution with F92D mutated Gads the cells responded similarly to Gads-deficient BMMCs (Example 5, FIGS. 12A-C and 13A-B).

Taken together, the present teachings clearly demonstrate that Gads SH2 dimerization via its amino acids F55, P56, W58, F59, E61, G62, A84-F92, V107-N111, Y115, F116, L125 and N126 is required for tight, preferentially paired binding of Gads to adjacent phospho-tyrosine motifs on LAT and is essential for antigen-induced activation in T cells and mast cells. Consequently, by identifying the residues which constitute the Gads dimerization interface it is possible to rationally design and screen for agents that bind to a pharmacophore binding site comprising these residues and inhibit Gads dimerization. Furthermore, the present teachings clearly demonstrate that Gads SH2 dimerization is stabilized by Gads N-terminal SH3 domain. Consequently, it is possible to rationally design and screen for an agent that binds to a pharmacophore binding site comprising the N-terminal SH3 domain to thereby inhibit Gads dimerization by allosterically reducing the stability of the SH2-mediated dimerization. These agents can further be used for inhibiting TCR-induced activation in T cells and/or FcεRI-induced activation in mast cells, in general, and for treating a disease associated with activation of T cells and/or an allergic response, in particular.

Thus, according to a first aspect of the present invention, there is provided an agent which inhibits Gads (SEQ ID NO: 1) dimerization, said agent interacting with a pharmacophore binding site comprising an amino acid selected from the group consisting of F55, P56, W58, F59, E61, G62, A84-F92, V107-N111, Y115, F116, L125 and N126 of SEQ ID NO: 1.

According to an alternative or an additional aspect of the present invention, there is provided an agent which inhibits Gads (SEQ ID NO: 1) dimerization, said agent interacting with a pharmacophore binding site comprising an amino acid sequence of an SH3 domain of SEQ ID NO: 1.

As used herein “Gads” also known as GRB2-related adaptor downstream of She and GRB2-related adapter protein 2, refers to a functional expression product of the GRAP2 gene. Full length Gads contains an SH2 domain flanked by two SH3 domains (N-terminus SH3 domain and C-terminus SH3 domain) and is capable of forming a dimer, binding phosphorylated LAT and binding SLP-76. A functional expression product of Gads refers to a Gads protein product capable of at least forming a dimer. Assays for testing binding and dimerization are well known in the art and include, but not limited to, size exclusion chromatography, fast protein liquid chromatography (FPLC), multi-angle light scattering (SEC-MALS) analysis, SDS-PAGE analysis, nano-DSF, yeast two-hybrid system (e.g. RRS) and flow cytometry.

According to specific embodiments, Gads is human Gads.

According to a specific embodiment, Gads amino acid sequence is as set forth in SEQ ID NO: 1, NP_001278754.1.

According to specific embodiments, Gads amino acid sequence is a splice variant of SEQ ID NO: 1.

According to a specific embodiment, Gads amino acid sequence is the SH2 domain of Gads, such as set forth in SEQ ID NO: 37.

As used herein, the phrase “inhibits dimerization” refers to the ability to interact with a pharmacophore binding site (a protein conformation which is essential for Gads dimerization) comprising an amino acid selected from the group consisting of F55, P56, W58, F59, E61, G62, A84-F92, V107-N111, Y115, F116, L125 and N126 of Gads (SEQ ID NO: 1) and thereby decrease Gads dimerization and/or a pharmacophore binding site comprising an amino acid sequence of a SH3 domain of Gads (SEQ ID NO: 1). The decrease is of at least 5% in Gads dimerization in the presence of the agent as compared to same in the absence of the agent. According to a specific embodiment, the decrease is in at least 10%, 20%, 30%, 40% or even higher say, 50%, 60%, 70%, 80%, 90%, 99% or even 100%. According to specific embodiments the decrease is at least 1.5 fold, at least 2 fold, at least 3 fold, at least 5 fold, at least 10 fold, or at least 20 fold as compared to same in the absence of the agent.

Dimerization of Gads can be assessed in multiple ways, including but not limited to size exclusion chromatography, fast protein liquid chromatography (FPLC), multi-angle light scattering (SEC-MALS) analysis, SDS-PAGE analysis, nano-DSF, yeast two-hybrid system (e.g. RRS) and flow cytometry, as further disclosed hereinabove and below and in the Examples section which follows.

Additionally or alternatively, as the dimerization of Gads is required for cooperative paired binding of Gads to adjacent phospho-tyrosine motifs on LAT, dimerization of Gads can be measured indirectly by assessing Gads binding to LAT. Methods of assessing binding are well known and are also disclosed hereinabove and in the Examples section which follows.

Additionally or alternatively, as the dimerization of Gads is required for antigen signaling in T cells and mast cells, dimerization of Gads can be measured indirectly by assessing T cell and/or mast cell activation. Methods of evaluating activation of T cells and mast cells are well known in the art, and are further disclosed in details hereinbelow and in the Examples section which follows.

The agent of the present invention inhibits dimerization on the protein level by interacting with a pharmacophore binding site of Gads (e.g. SEQ ID NO: 1).

As used herein, the term “pharmacophore” refers to a molecular structure within Gads that is responsible for dimerization.

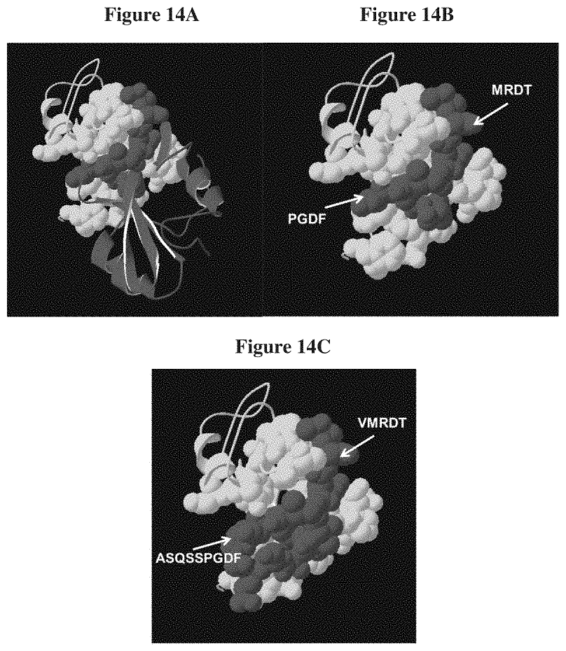

A pharmacophore may be used to design or select for an agent that binds the pharmacophore binding site of Gads and inhibit Gads dimerization. For example, as shown in FIG. 5F, R109 and F92 which are found at the center of the dimerization interface flank two pockets (marked by arrows). Peptides such as PGDF (SEQ ID NO: 33) and MRDT (SEQ ID NO: 34) from adjacent Gads SH2 occupy these two pockets (FIGS. 14A-B). An agent that mimics any of these peptides can block their binding pockets to thereby inhibit Gads dimerization. Extended peptides, encompassing the entire core dimerization interface (see red residues in FIG. 5C), ASQSSPGDF (SEQ ID NO: 35) and VMRDT (SEQ ID NO: 36) are shown in FIG. 14C. An agent that mimics these peptides can inhibit Gads dimerization, also known as a competitive inhibitor. Additionally or alternatively, an agent that binds the SH3 domain of Gads can allosterically reduce the stability of the SH2-mediated dimerization thereby inhibit Gads dimerization.

Consequently, the agent of the present invention inhibits dimerization by interacting with a pharmacophore binding site of Gads.

The phrase “amino acid of Gads” is intended to encompass an amino acid residue specifically identified, as by, e.g., reference to a residue along with a SEQ ID NO (e.g. SEQ ID NO: 1), as well as amino acid residues occupying analogous positions in related proteins. A related protein refers to a functional expression product of Gads as defined herein, which can be derived from the same organism or from a different organism from the organism from which the reference protein is derived.

According to specific embodiments, the pharmacophore binding site comprises an amino acid selected from the group consisting of F55, P56, W58, F59, E61, G62, A84-F92, V107-N111, Y115, F116, L125 and N126 of SEQ ID NO: 1, each possibility represents a separate embodiment of the present invention.

According to specific embodiments, the pharmacophore binding site comprises an amino acid selected from the group consisting of A84-F92 and V107-N111, each possibility represents a separate embodiment of the present invention.

According to specific embodiments, the pharmacophore binding site comprises F92 and/or R109 of SEQ ID NO: 1.

According to specific embodiments, the pharmacophore binding site comprises an amino acid sequence of an SH3 domain of Gads (SEQ ID NO: 1).

As used herein, the term “SH3 domain of Gads” refers to SEQ ID NO: 50.

According to a specific embodiment, the agent binds the hydrophobic surface found on the SH3 domain of Gads (SEQ ID NO: 50).

According to specific embodiments, the interaction of the agent with the pharmacophore binding site is covalent. According to other specific embodiments, the interaction of the agent with the pharmacophore binding site is non-covalent, wherein the juxtaposition is energetically favored by hydrogen bonding or van der Waals or electrostatic interactions. According to specific embodiments, the interaction is reversible. According to other specific embodiments, the interaction in irreversible.

According to specific embodiments, the agent interacts with at least one amino acid residues of Gads, as specified herein.

According to other specific embodiments, the agent interacts with at least two, at least 3, at least 4, at least 5, at least 7, at least 10 or at least 14 amino acid residues of Gads, as specified herein.

According to a specific embodiments, the agent interacts with an amino acid residue F92 and/or R109 of Gads (SEQ ID NO: 1).

Following is a non-limiting list of agents capable of inhibiting Gads dimerization.

According to specific embodiments, the agent is a peptide.

Non-limiting Examples of such a peptide include peptides comprising an amino acid sequence selected from the group consisting of PGDF (SEQ ID NO: 33), MRDT (SEQ ID NO: 34), MRDN (SEQ ID NO: 38), PGDFGVMRD (SEQ ID NO: 39), PGDFGGVMRD (SEQ ID NO: 40), PGDFPVMRD (SEQ ID NO: 41), ASQSSPGDF (SEQ ID NO: 35), VMRDT (SEQ ID NO: 36), VMRDN (SEQ ID NO: 42) and ASQSSPGDFGVMRD (SEQ ID NO: 43), each possibility represents a separate embodiments of the present invention.

According to specific embodiments, the peptides are no more than 100, no more than 50, no more than 25 or no more than 10 amino acids in length (e.g., not including the length of the cell penetrating peptide as described below).

According to specific embodiments, the peptide is at least 4 amino acids in length.

According to a specific embodiments, the peptide comprises an amino acids sequence consisting of an amino acid sequence selected from the group consisting of PGDF (SEQ ID NO: 33), MRDT (SEQ ID NO: 34), MRDN (SEQ ID NO: 38), PGDFGVMRD (SEQ ID NO: 39), PGDFGGVMRD (SEQ ID NO: 40), PGDFPVMRD (SEQ ID NO: 41), ASQSSPGDF (SEQ ID NO: 35), VMRDT (SEQ ID NO: 36), VMRDN (SEQ ID NO: 42) and ASQSSPGDFGVMRD (SEQ ID NO: 43), each possibility represents a separate embodiments of the present invention.

According to a specific embodiments, the peptide consists of an amino acid sequence selected from the group consisting of PGDF (SEQ ID NO: 33), MRDT (SEQ ID NO: 34), MRDN (SEQ ID NO: 38), PGDFGVMRD (SEQ ID NO: 39), PGDFGGVMRD (SEQ ID NO: 40), PGDFPVMRD (SEQ ID NO: 41), ASQSSPGDF (SEQ ID NO: 35), VMRDT (SEQ ID NO: 36), VMRDN (SEQ ID NO: 42) and ASQSSPGDFGVMRD (SEQ ID NO: 43), each possibility represents a separate embodiments of the present invention.

According to specific embodiments, the peptide is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the a peptide comprising an amino acid sequence selected from the group consisting of PGDF (SEQ ID NO: 33), MRDT (SEQ ID NO: 34), MRDN (SEQ ID NO: 38), PGDFGVMRD (SEQ ID NO: 39), PGDFGGVMRD (SEQ ID NO: 40), PGDFPVMRD (SEQ ID NO: 41), ASQSSPGDF (SEQ ID NO: 35), VMRDT (SEQ ID NO: 36), VMRDN (SEQ ID NO: 42) and ASQSSPGDFGVMRD (SEQ ID NO: 43), as long as the function (e.g. inhibiting Gads dimerization) is maintained.

Sequence identity can be determined using any protein sequence alignment algorithm such as Blast and ClustalW.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO-CH2-), sulfinylmethylene bonds (—S(═O)—CH2-), α-aza bonds (—NH—N(R)—CO—), wherein R is any alkyl (e.g., methyl), amine bonds (˜CH2-NH—), sulfide bonds (˜CH2-S—), ethylene bonds (˜CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino acid | Code |
|---|---|
| ornithine | Orn |
| α-aminobutyric acid | Abu |
| D-alanine | Dala |
| D-arginine | Darg |
| D-asparagine | Dasn |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-N-methylalanine | Dnmala |
| D-N-methylarginine | Dnmarg |
| D-N-methylasparagine | Dnmasn |
| D-N-methylasparatate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| D-N-methylmethionine | Dnmmet |
| D-N-methylornithine | Dnmorn |
| D-N-methylphenylalanine | Dnmphe |
| D-N-methylproline | Dnmpro |
| D-N-methylserine | Dnmser |
| D-N-methylthreonine | Dnmthr |
| D-N-methyltryptophan | Dnmtrp |
| D-N-methyltyrosine | Dnmtyr |
| D-N-methylvaline | Dnmval |
| L-norleucine | Nle |
| L-norvaline | Nva |
| L-ethylglycine | Etg |
| L-t-butylglycine | Tbug |
| L-homophenylalanine | Hphe |
| α-naphthylalanine | Anap |
| penicillamine | Pen |
| γ-aminobutyric acid | Gabu |
| cyclohexylalanine | Chexa |
| cyclopentylalanine | Cpen |
| α-amino-α-methylbutyrate | Aabu |
| α-aminoisobutyric acid | Aib |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methyl glutamic acid | Dmglu |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmtyr |
| D-α-methylvaline | Dmval |
| N-cyclobutylglycine | Ncbut |
| N-cycloheptylglycine | Nchep |
| N-cyclohexylglycine | Nchex |
| N-cyclodecylglycine | Ncdec |
| N-cyclododecylglycine | Ncdod |
| N-cyclooctylglycine | Ncoct |
| N-cyclopropylglycine | Ncpro |
| N-cycloundecylglycine | Ncund |
| N-(2-aminoethyl)glycine | Naeg |
| N-(2,2-diphenylethyl)glycine | Nbhm |
| N-(3,3-diphenylpropyl)glycine | Nbhe |
| 1-carboxy-1-(2,2-diphenyl | Nmbc |
| ethylamino)cyclopropane | |
| phosphoserine | pSer |
| phosphotyrosine | pTyr |
| 2-aminoadipic acid | |
| hydroxyproline | Hyp |
| aminonorbornyl-carboxylate | Norb |
| aminocyclopropane-carboxylate | Cpro |
| N-(3-guanidinopropyl)glycine | Narg |
| N-(carbamylmethyl)glycine | Nasn |
| N-(carboxymethyl)glycine | Nasp |
| N-(thiomethyl)glycine | Ncys |
| N-(2-carbamylethyl)glycine | Ngln |
| N-(2-carboxyethyl)glycine | Nglu |
| N-(imidazolylethyl)glycine | Nhis |
| N-(1-methylpropyl)glycine | Nile |
| N-(2-methylpropyl)glycine | Nleu |
| N-(4-aminobutyl)glycine | Nlys |
| N-(2-methylthioethyl)glycine | Nmet |
| N-(3-aminopropyl)glycine | Norn |
| N-benzylglycine | Nphe |
| N-(hydroxymethyl)glycine | Nser |
| N-(1-hydroxyethyl)glycine | Nthr |
| N-(3-indolylethyl) glycine | Nhtrp |
| N-(p-hydroxyphenyl)glycine | Ntyr |
| N-(1-methylethyl)glycine | Nval |
| N-methylglycine | Nmgly |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylnorleucine | Nmnle |
| L-N-methylnorvaline | Nmnva |
| L-N-methyl-ethylglycine | Nmetg |
| L-N-methyl-t-butylglycine | Nmtbug |
| L-N-methyl-homophenylalanine | Nmhphe |
| N-methyl-α-naphthylalanine | Nmanap |
| N-methylpenicillamine | Nmpen |
| N-methyl-γ-aminobutyrate | Nmgabu |
| N-methyl-cyclohexylalanine | Nmchexa |
| N-methyl-cyclopentylalanine | Nmcpen |
| N-methyl-α-amino-α-methylbutyrate | Nmaabu |
| N-methyl-α-aminoisobutyrate | Nmaib |
| L-α-methylarginine | Marg |
| L-α-methylasparagine | Masn |
| L-α-methylaspartate | Masp |
| L-α-methylcysteine | Mcys |
| L-α-methylglutamine | Mgln |
| L-α-methylglutamate | Mglu |
| L-α-methylhistidine | Mhis |
| L-α-methylisoleucine | Mile |
| L-α-methylleucine | Mleu |
| L-α-methyllysine | Mlys |
| L-α-methylmethionine | Mmet |
| L-α-methylornithine | Morn |
| L-α-methylphenylalanine | Mphe |
| L-α-methylproline | Mpro |
| L-α-methylserine | Mser |
| L-α-methylthreonine | Mthr |
| L-α-methyltryptophan | Mtrp |
| L-α-methyltyrosine | Mtyr |
| L-α-methylvaline | Mval |
| L-α-methylnorvaline | Mnva |
| L-α-methylethylglycine | Metg |
| L-α-methyl-t-butylglycine | Mtbug |
| L-α-methyl-homophenylalanine | Mhphe |
| α-methyl-α-naphthylalanine | Manap |
| α-methylpenicillamine | Mpen |
| α-methyl-γ-aminobutyrate | Mgabu |
| α-methyl-cyclohexylalanine | Mchexa |
| α-methyl-cyclopentylalanine | Mcpen |
| N-(N-(2,2-diphenylethyl) | Nnbhm |
| carbamylmethyl-glycine | |
| N-(N-(3,3-diphenylpropyl) | Nnbhe |
| carbamylmethyl-glycine | |
| 1,2,3,4-tetrahydroisoquinoline-3- | Tic |
| carboxylic acid | |
| phosphothreonine | pThr |
| O-methyl-tyrosine | |
| hydroxylysine | |

The peptides of some embodiments of the invention are preferably utilized in a linear form, although it will be appreciated that in cases where cyclicization does not severely interfere with peptide characteristics, cyclic forms of the peptide can also be utilized.

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

According to specific embodiments, the peptides comprise phosphorylated residues e.g. serine phosphorylated residues.

The amino acids of the peptides of the present invention may be substituted either conservatively or non-conservatively.

According to specific embodiments, the substitutions are determined by computational peptide docking.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH [(—CH₂)₅—COOH]—CO— for aspartic acid. Those non-conservative substitutions which fall under the scope of the present invention are those which still constitute a peptide having dimerization-inhibitory properties.

The N and C termini of the peptides of the present invention may be protected by functional groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

The peptides of the present invention may be attached (either covalently or non-covalently) to a penetrating agent.

As used herein the phrase “penetrating agent” refers to an agent which enhances translocation of any of the attached peptide across a cell membrane.

According to one embodiment, the penetrating agent is a peptide and is attached to the peptide (either directly or non-directly) via a peptide bond.

Typically, peptide penetrating agents have an amino acid composition containing either a high relative abundance of positively charged amino acids such as lysine or arginine, or have sequences that contain an alternating pattern of polar/charged amino acids and non-polar, hydrophobic amino acids.

According to other specific embodiments of the invention, the peptide is attached to a non-proteinaceous moiety.

According to specific embodiments, the peptide and the attached non-proteinaceous moiety are covalently attached, directly or through a spacer or a linker.

The phrase “non-proteinaceous moiety” as used herein refers to a molecule not including peptide bonded amino acids that is attached to the above-described peptide. According to a specific embodiment the non-proteinaceous is a non-toxic moiety. Exemplary non-proteinaceous moieties which may be used according to the present teachings include, but are not limited to a drug, a chemical, a small molecule, a polynucleotide, a detectable moiety, polyethylene glycol (PEG), Polyvinyl pyrrolidone (PVP), poly(styrene comaleic anhydride) (SMA), and divinyl ether and maleic anhydride copolymer (DIVEMA). According to specific embodiments of the invention, the non-proteinaceous moiety comprises polyethylene glycol (PEG).

The peptides of some embodiments of the invention may be synthesized and purified by any techniques that are known to those skilled in the art of peptide synthesis, such as, but not limited to, solid phase and recombinant techniques.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50.

According to specific embodiments, the agent is a small molecule.

According to specific embodiments, the agent is a small molecule which can be identified according to the screening method provided hereinbelow.

According to specific embodiments, the agent is a known SH3 inhibitor e.g. but not limited to dirhodium conjugates, benzoquinoline derivatives, pseudoprolines (WPro) and/or such disclosed in Lu et al. Curr Med Chem. 2010; 17(12):1117-24, the contents of which are fully incorporated herein by reference.

According to other specific embodiments, the agent is an antibody. Preferably, the antibody specifically binds at least one epitope of a chromophore binding site of Gads. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof (such as Fab, F(ab′)2, Fv, scFv, dsFv, or single domain molecules such as VH and VL) that are capable of binding to an epitope of an antigen.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) disulfide-stabilized Fv (“dsFv”), a genetically engineered antibody including the variable region of the light chain and the variable region of the heavy chain, linked by a genetically engineered disulfide bond.

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vii) Single domain antibodies or nanobodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

As Gads is localized intracellularly, an antibody or antibody fragment capable of specifically binding Gads is typically an intracellular antibody (also known as “intrabodies”). Intracellular antibodies are essentially SCA to which intracellular localization signals have been added (e.g., ER, mitochondrial, nuclear, cytoplasmic). This technology has been successfully applied in the art (for review, see Richardson and Marasco, 1995, TIBTECH vol. 13). Intrabodies have been shown to virtually eliminate the expression of otherwise abundant cell surface receptors and to inhibit a protein function within a cell (See, for example, Richardson et al., 1995, Proc. Natl. Acad. Sci. USA 92: 3137-3141; Deshane et al., 1994, Gene Ther. 1: 332-337; Marasco et al., 1998 Human Gene Ther 9: 1627-42; Shaheen et al., 1996 J. Virol. 70: 3392-400; Werge, T. M. et al., 1990, FEBS Letters 274:193-198; Carlson, J. R. 1993 Proc. Natl. Acad. Sci. USA 90:7427-7428; Biocca, S. et al., 1994, Bio/Technology 12: 396-399; Chen, S-Y. et al., 1994, Human Gene Therapy 5:595-601; Duan, L et al., 1994, Proc. Natl. Acad. Sci. USA 91:5075-5079; Chen, S-Y. et al., 1994, Proc. Natl. Acad. Sci. USA 91:5932-5936; Beerli, R. R. et al., 1994, J. Biol. Chem. 269:23931-23936; Mhashilkar, A. M. et al., 1995, EMBO J. 14:1542-1551; PCT Publication No. WO 94/02610 by Marasco et al.; and PCT Publication No. WO 95/03832 by Duan et al.).

To prepare an intracellular antibody expression vector, the cDNA encoding the antibody light and heavy chains specific for the target protein of interest are isolated, typically from a hybridoma that secretes a monoclonal antibody specific for the marker. Hybridomas secreting anti-marker monoclonal antibodies, or recombinant monoclonal antibodies, can be prepared using methods known in the art. Once a monoclonal antibody specific for the marker protein is identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process and the nucleotide sequences of antibody light and heavy chain genes are determined. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database. Once obtained, the antibody light and heavy chain sequences are cloned into a recombinant expression vector using standard methods.

For cytoplasmic expression of the light and heavy chains, the nucleotide sequences encoding the hydrophobic leaders of the light and heavy chains are removed. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In another embodiment, the vector encodes a single chain antibody (scFv) wherein the variable regions of the light and heavy chains are linked by a flexible peptide linker [e.g., (Gly₄Ser)₃ and expressed as a single chain molecule. To inhibit marker activity in a cell, the expression vector encoding the intracellular antibody is introduced into the cell by standard transfection methods, as discussed hereinbefore.

Once antibodies are obtained, they may be tested for activity, for example via ELISA.

Another agent which can be used along with some embodiments of the invention is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Agents that can be used according to the present teachings can be identified from various screening methods known in the art.

Alternatively or additionally, the present teachings are directed to the identification of agents as according to the following aspect.

Thus, according to another aspect of the present invention there is provided a method of identifying an agent that inhibits Gads dimerization, the method comprising:

(a) designing a test agent which inhibits Gads (SEQ ID NO: 1) dimerization by interacting with a pharmacophore binding site comprising an amino acid selected from the group consisting of F55, P56, W58, F59, E61, G62, A84-F92, V107-N111, Y115, F116, L125 and N126 of SEQ ID NO: 1; and optionally

(b) testing an effect of said agent on Gads dimerization or a biological outcome thereof.

According to another aspect of the present invention there is provided a method of identifying an agent that inhibits Gads dimerization, the method comprising:

(a) designing a test agent which inhibits Gads (SEQ ID NO: 1) dimerization by interacting with a pharmacophore binding site comprising an amino acid sequence of a SH3 domain of SEQ ID NO: 1; and optionally

(b) testing an effect of said agent on Gads dimerization or a biological outcome thereof.

As used herein, the phrase “designing a test agent” includes an agent developed de-novo, a known agent or a modified known agent.

Methods for designing a test agent of the present invention are known in the art and have been described for example in International Patent Application Publication No: WO2002046392, US Patent Application Publication No: US20060128699; Chinese Patent No. CN101329698A, Van Antwerpen et al. Free Radic Res. 2015 June; 49(6):711-20, Macalino et al. Arch Pharm Res. 2015 September; 38(9):1686-701 and Mavromoustakos et al. Curr Med Chem. 2011; 18(17):2517-30, each of which is incorporated herein by reference.

Hence, for example, according to some embodiment of the invention, the agent is selected based on in-silico prediction using e.g. structural model of the Gads SH2 dimerization interface e.g. the two adjacent murine Gads SH2 units from PDB file 1R1P.

Once a suitable agent is identified it is synthesized and may be further qualified using a functional testing its effect on Gads dimerization or a biological outcome thereof.

According to specific embodiments, the testing is effected effect in-vitro or ex-vivo.

According to other specific embodiments, the testing is effected in-vivo.

As noted, the effect on Gads dimerization can be assessed in multiple ways well known in the art including those described hereinabove and below and in the Examples section which follows.

Thus, according to specific embodiments, the method further comprising providing said test agent and determining Gads dimerization in the presence of said test agent, wherein a decrease in said Gads dimerization in the presence of said test agent below a predetermined threshold as compared to same in the absence of said test agent indicates said agent inhibits Gads dimerization.

According to specific embodiments, the predetermined threshold is of at least 5%, at least 10%, 20%, 30%, 40% or even higher say, 50%, 60%, 70%, 80%, 90%, 99% or even 100% as compared to same in the absence of the agent. According to specific embodiments the predetermined threshold is at least 1.5 fold, at least 2 fold, at least 3 fold, at least 5 fold, at least 10 fold, or at least 20 fold as compared to same in the absence of the agent.

According to specific embodiments, the functional assay is based on inducible dimerization of Gads that occurs upon cooperatively paired binding to a dual-phosphorylated LAT peptide. An agent of interest inhibits cooperatively paired binding of Gads to a dual phosphorylated LAT peptide (2pY-LAT) by inhibiting Gads dimerization, but does not markedly affect the non-cooperative binding of Gads to a single-phosphorylated LAT peptide (pY171-LAT).

The following non-limiting screening methods can be used:

1. FRET-based assay system: Two pools of Gads protein are labeled with different fluorescent labels that are capable of exhibiting FRET when brought into proximity. Addition of the dual-phosphorylated 2pY-LAT peptide induces dimerization, resulting in increased FRET. Hence, tested agents are tested for an agent that disrupts the 2pY-LAT-induced FRET signal by inhibiting dimerization. Alternatively, one pool is labeled with a fluorescent label and the other with a quencher. 2pY-LAT peptide induces dimerization, which is detected as fluorescence quenching. An agent of interest inhibits quenching by inhibiting dimerization.

2. Split-luciferase-based assay system: Recombinant Gads protein are expressed while fused by a flexible linker to the N- or C-terminal lobes of the luciferase enzyme. 2pY-LAT-induced Gads dimerization brings the two halves of the luciferase enzyme into proximity, reconstituting luciferase activity, which is measured by the production of light in the presence of ATP and an appropriate luciferase substrate. An agent of interest disrupts 2pY-LAT-induced Gads dimerization. Alternatively, the N-luciferase- and C-luciferase-fused Gads constructs are expressed in intact T cells or mast cells, where basal and antigen-induced Gads dimerization are measured by the resulting luciferase activity and an agent of interest decreases the basal or antigen-induced increase in luciferase activity.

3. Bead-based assay system: Biotinylated LAT peptides, either single or dual-phosphorylated, are bound to unlabeled or differentially labeled streptavidin microbeads; and incubated with fluorescent Gads protein. The ability of the fluorescent Gads protein to bind preferentially to the 2pY-LAT beads is assessed by FACS or using a high-throughput fluorescent plate screener. In this assay, competitive binding of Gads to single- or dual-phosphorylated beads can be imaged, wherein an agent of interest decreases the selectivity of Gads for the dual-phosphorylated beads. Alternatively, a biotinylated phospho-LAT peptide is bound to the surface of a multi-well plate and a competitive binding assay is performed to assess the ability of a soluble phospho-LAT peptide to competitively inhibit Gads binding to the plate-bound peptide. Wherein an agent of interest decreases the selectivity of Gads for dual-phosphorylated competitor peptide, as compared to single-phosphorylated competitor peptide.

According to specific embodiments, each of the screening assays is performed using at least one of the following Gads protein constructs: SH2 alone or full length Gads, either wild type or bearing mutations in the dimerization domain (e.g. F92D or F92A/R109A). Typically, the SH2 domain alone is used in the first step, in order to identify compounds that specifically inhibit Gads SH2 dimerization; and at subsequent steps, the agent is validated using full length Gads, to verify that the agent is capable of inhibiting the function of the full length Gads protein.

As shown in the Examples section which follows, cooperatively paired binding of Gads is impaired by mutations, such as F92D or F92A/R109A, that impair spontaneous Gads SH2 dimerization. Hence, the results of the screening assays can be validated, by verifying that the selected agent causes wild type Gads protein to mimic the behavior of the dimerization-defective Gads protein.

Thus, according to specific embodiments, the method further comprising providing said test agent and determining Gads dimerization in the presence of said test agent, wherein a decrease in said Gads dimerization in the presence of said test agent to a level that is comparable to dimerization of F92D mutated Gads and/or F92A/R109A mutated Gads in the absence of said test agent indicates said agent inhibits Gads dimerization.

According to some embodiments, candidate agents selected according to the methods described above are tested for their biological activity, specificity and toxicity in-vitro in cell cultures or in-vivo in e.g. allergy, autoimmunity, cancer and inflammation models.

According to specific embodiments, the method comprising providing said test agent and testing its inhibitory effect on mast cells and/or T cells activation. Such assays are well known in the art and are further described in details hereinbelow and in the Examples section which follows.

According to specific embodiments, the method comprising providing said test agent and testing an anti-allergic activity of same.

Thus, for example, in-vitro testing can be effected in primary murine bone-marrow derived mast cells grown in culture, sensitized with antigen-specific IgE, and then stimulated with the antigen recognized by the IgE, which activates the cells via their FcεRI. FcεRI-induced responses are measured, including, but not limited to degranulation, expression of surface markers, calcium flux or cytokine production.

An exemplary in vivo assay includes, but is not limited to Passive cutaneous anaphylaxis, in which animals are sensitized by intradermal injection of antigen-specific IgE, for example to the ear. Antigen is then applied intravenously, to stimulate resident mast cells via their FcεRI. Physiologic consequences of FcεRI activation are measured, for example, ear swelling or plasma leakage into the tissues. Additional non-limiting in vivo assays include passive systemic anaphylaxis, in which mice are sensitized intravenously with antigen-specific IgE, and subsequent application of antigen induces an anaphylactic response, which can be measured by measuring heart rate, histamine release, survival, and other responses.

According to other specific embodiments, the method comprising providing said test agent and testing an anti-autoimmune activity of same. Non-limiting examples of autoimmunity models include the EAE mouse (a well-known model of multiple sclerosis) and the NOD mouse (a well-known model of diabetes type I).

According to other specific embodiments, the method comprising providing said test agent and testing an anti-cancer and/or anti-inflammation activity of same. Non-limiting examples of cancer models include TCR-transgenic mice bearing a T cell specific for a known antigen. These T cells are then transferred into a mouse that bears a tumor expressing the specific antigen. Following, the effect of the agent on the anti-tumor response of the T cells in the recipient mouse is determined.

A non-limiting example of chronic inflammation model include inflammatory bowel disease (IBD) such as disclosed for example in Low et al. [Drug Des Devel Ther. 2013; 7: 1341-1357], the contents of which are fully incorporated herein by reference.

Examples 4-5, in the Examples section which follows, clearly demonstrate that Gads SH2 dimerization via its amino acids F55, P56, W58, F59, E61, G62, A84-F92, V107-N111, Y115, F116, L125 and N126 is required for antigen signaling in T cells and in mast cells. Therefore, the present teachings suggest that by inhibiting Gads dimerization, the agents of the present invention impair formation of the complete LAT signalosome in T cells and/or mast cells, and thereby block their activation.

Thus, according to another aspect of the present invention, there is provided a method of inhibiting activation of a T cell and/or a mast cell, the method comprising contacting the T cell and/or the mast cell with the agent of the present invention, thereby inhibiting activation of the T cell and/or the mast cell.

As used herein, the term “mast cell (MC)” refers to a highly granulated cell containing numerous granules comprising substances such as histamine and heparin. Typical markers of MCs include but are not limited to CD117 (c-Kit) and FcεRI.

As used herein the term “activation of a mast cell” refers to the process of stimulating mast cell that results in at least one of the following processes: calcium flux, growth, maturation, proliferation, migration, survival, apoptosis, degranulation, mediator release, priming (preparing the cell for action, alerting it to standby), chemotaxis, adherence and synthesis and secretion of cytokines, growth factors, arachidonic acid metabolites, chemokines, phospholipid metabolites and others.

According to specific embodiments, activation of the mast cell results in at least one of: calcium flux; degranulation; and cytokine production and/or secretion.

As Gads is part of the FcεRI signaling cascade in mast cells, according to specific embodiments, mast cell activation is FcεRI dependent.

As used herein, the term “FcεRI”, also known as “high-affinity IgE receptor”, refers to an antigen receptor for the Fc region of immunoglobulin E (IgE) present on the surface of mast cells and may comprise the FcεRIα, FcεRIβ and/or the FcεRIγ chain. Crosslinking of the FcεRI via IgE-antigen complexes triggers ITAM-dependent signaling cascades, initiated by Src- and Syk-family tyrosine kinases.

Methods of monitoring activation and/or inhibition of activation of a MC are known in the art and are also described in the Examples section which follows. Non-limiting examples include assays which evaluate cell viability and survival such as the MTT test which is based on the selective ability of living cells to reduce the yellow salt MTT (3-(4, 5-dimethylthiazolyl-2)-2, 5-diphenyltetrazolium bromide) (Sigma, Aldrich St Louis, Mo., USA) to a purple-blue insoluble formazan precipitate; the Annexin V assay [ApoAlert® Annexin V Apoptosis Kit (Clontech Laboratories, Inc., CA, USA)]; the Senescence associated-β-galactosidase assay (Dimri G P, Lee X, et al. 1995. A biomarker that identifies senescent human cells in culture and in aging skin in vivo. Proc Natl Acad Sci USA 92:9363-9367); the TUNEL assay [Roche, Mannheim, Germany]; Assays which evaluate cell proliferation capacity such as the BrdU incorporation assay [Cell Proliferation ELISA BrdU colorimetric kit (Roche, Mannheim, Germany)]; assay which evaluate intracellular calcium concentration; assays which evaluate production and/or secretion of cytokines and other mediators such as intracellular staining (e.g. anti-CD63, anti-CD107a, anti-IL-6), ELISPOT and ELISA (e.g. IL-6 ELISA kit (R&D, Abcam), PGD₂ ELISA kit (Cayman chemicals); GM-CSF ELISA kit (Peprotech); colorimetric and fluorometric enzymatic assays based on incubation with the specific mediator's susbstrate (e.g. Bachelet et al. J. Immunol. (2005) 175:7989-95; and assay [Gibbs et al. Clin Exp Allergy (2008) 38:480-5]; as well as various RNA and protein detection methods, such as evaluating expression of molecules involved in the signaling cascade using e.g. PCR, Western blot, immunopercipitation and immunohistochemistry; evaluating the level of phosphorylation on tyrosine residues on key signal molecules such as the kinases syk, lyn, fyn, erk and the phospholipase PLC-γ1. The higher the level of phosphorylation, the higher the MC activation is. This can be detected by flow cytometry analysis or by Western blot analysis using anti specific anti-phosphotyrosine antibodies for the same molecules.

As used herein, the term “T cells” refers to differentiated lymphocytes with a CD3⁺, T cell receptor (TCR)⁺ having either CD4⁺ or CD8⁺ phenotype. The T cell may be either an effector or a regulatory T cell.

According to specific embodiments, the T cell is an effector T cell.

As used herein, the term “effector T cells” refers to a T cell that activates or directs other immune cells e.g. by producing cytokines or has a cytotoxic activity e.g., CD4⁺, Th1/Th2, CD8⁺ cytotoxic T lymphocyte.

According to other specific embodiments, the T cell is a regulatory T cell.

As used herein, the term “regulatory T cell” or “Treg” refers to a T cell that negatively regulates the activation of other T cells, including effector T cells, as well as innate immune system cells. Treg cells are characterized by sustained suppression of effector T cell responses. According to a specific embodiment, the Treg is a CD4⁺CD25⁺Foxp3⁺ T cell.

According to specific embodiments, the T cells are CD4⁺ T cells.

According to other specific embodiments, the T cells are CD8⁺ T cells.

According to specific embodiments, the T cells are memory T cells. Non-limiting examples of memory T cells include effector memory CD4⁺ T cells with a CD3⁺/CD4⁺/CD45RA⁻/CCR7⁻ phenotype, central memory CD4⁺ T cells with a CD3⁺/CD4⁺/CD45RA⁻/CCR7⁺ phenotype, effector memory CD8⁺ T cells with a CD3⁺/CD8⁺/CD45RA⁻/CCR7⁻ phenotype and central memory CD8⁺ T cells with a CD3⁺/CD8⁺/CD45RA⁻/CCR7⁺ phenotype.

As used herein the term “activation of a T cell” refers to the process of stimulating T cell that results in at least one of the following processes: calcium flux, growth, maturation, differentiation, proliferation, migration, survival, adherence and synthesis and secretion of cytokines, growth factors and others, expression of activation markers and induction of regulatory or effector functions.

According to specific embodiments, activation of the T cell results in at least one of: expression of activation markers; and phosphorylation of PLC-γ1.

As Gads is part of the TCR signaling cascade in T cells, according to specific embodiments, T cell activation is TCR dependent.

As used herein the term “TCR” or “T cell receptor” refers to an antigen-recognition molecule present on the surface of T cells and may comprise the TCRα chain, the TCRβ chain, the TCRγ chain and/or the TCRδ chain. Activation of a TCR results in ITAM-dependent signaling cascades, initiated by Src- and Syk-family tyrosine kinases.

Methods of monitoring activation and/or inhibition of activation of a T cell are known in the art and are also described in the Examples section which follows. Non-limiting examples include assays which evaluate cell viability and survival such as the MTT test, the Annexin V assay, the Senescence associated-β-galactosidase assay and the TUNEL assay; assays which evaluate cell proliferation capacity such as the BrdU incorporation assay; assay which evaluate intracellular calcium concentration; assays which evaluate production and secretion of cytokines (e.g. INFγ, IL-6, IL-4, IL-2) such as intracellular staining, ELISPOT and ELISA [e.g. IL-6 ELISA kit (R&D, Abcam), IL-2 ELISA kit (R&D, Abcam), IL-4 ELISA kit (R&D, Abcam)]; cytotoxicity assays such as chromium release; assays which evaluate expression of activation markers such as CD25 and CD69 using e.g. flow cytometry; as well as various RNA and protein detection methods, such as evaluating expression of molecules involved in the signaling cascade using e.g. PCR, Western blot, immunopercipitation and immunohistochemistry; evaluating the level of phosphorylation on tyrosine residues on key signal molecules such as the kinases syk, lyn, fyn, erk and the phospholipase PLC-γ1. The higher the level of phosphorylation, the higher the T cell activation is. This can be detected by flow cytometry analysis or by Western blot analysis using specific anti-phosphotyrosine antibodies for the same molecules.

According to specific embodiments, determining T cell activation is effected in-vitro or ex-vivo e.g. in a mixed lymphocyte reaction (MLR).

As used herein the term “inhibiting activation” refers to a statistically significant decrease in activation, e.g., as defined hereinabove, as compared to a control cell (the respective T cells or mast cell) being under the same assay conditions without the treatment with the agent.

According to specific embodiments inhibiting activation refers both to suppressing activation and to preventing activation.

According to a specific embodiment, inhibiting activation is by at least 5%, 10%, 20%, 30%, 50%, 80%, 90%, 95% and even 100% as compared to the activation in the control cell. According to specific embodiments the decrease is at least 1.5 fold, at least 2 fold, at least 3 fold, at least 5 fold, at least 10 fold, or at least 20 fold as compared to the activation in the control cell.

According to a specific embodiment, contacting with the agent is effected in-vitro.

According to another specific embodiment, contacting is effected ex-vivo.

According to another specific embodiment, contacting is effected in-vivo.

As the agents of the present invention can block mast cell and/or T cell activation they can be used in clinical settings.

Thus, according to another aspect of the present invention, there is provided a method of treating or preventing an allergic response in a subject in need thereof, the method comprising administering to the subject a therapeutically effective amount of the agent disclosed herein, thereby treating or preventing the allergic response in the subject.

According to another aspect of the present invention, there is provided an agent as disclosed herein, for use in the treatment or prevention of an allergic response.

According to another aspect of the present invention, there is provided a method of treating or preventing a disease associated with activation of T cells in a subject in need thereof, the method comprising administering to the subject a therapeutically effective amount of the agent disclosed herein, thereby treating or preventing the disease associated with activation of T cells in the subject.

According to another aspect of the present invention, there is provided an agent as disclosed herein, for use in the treatment or prevention of a disease associated with activation of T cells.

As used herein, the term “treating” refers to inhibiting, preventing or arresting the development of a pathology (e.g. allergy, autoimmune disease, chronic inflammation, cancer) and/or causing the reduction, remission, or regression of a pathology. Those of skill in the art will understand that various methodologies and assays can be used to assess the development of a pathology or reduction, remission or regression of a pathology, as further disclosed herein.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings, at any age and of any gender which suffer from the pathology. Preferably, this term encompasses individuals who are at risk to develop the pathology.

According to specific embodiments, the disorder is an allergic response or allergy. Specific examples of allergic response which may be treated according to the teachings of the present invention include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

According to specific embodiments, the disorder is a disease associated with activation of T cells.

As used herein, “a disease associated with activation of T cells” refers to a pathological condition which onset or progression is associated with over activity of T cells and can be benefited from inhibiting T cells activity. The disease can be associated with activation of effector T cells or regulatory T cells.

This includes chronic and acute disorders or diseases including those pathological conditions which predispose the mammal to the disorder in question. Non-limiting examples of disorders to be treated herein include autoimmune diseases, graft rejection disease (e.g. graft vs. host disease), cancer e.g. benign and malignant tumors; leukemias and lymphoid malignancies; neuronal, glial, astrocytal, hypothalamic and other glandular, macrophagal, epithelial, stromal and blastocoelic disorders; inflammatory disorders including chronic inflammation, angiogenic, immunologic disorders or hyperpermeability states.

According to specific embodiments, the disease is an autoimmune disease. Specific examples of autoimmune diseases which may be treated according to the teachings of the present invention include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), Chronic obstructive pulmonary disease (COPD), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), Crohn's disease, ulcerative colitis, psoriasis autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

According to other specific embodiments, the disease is a transplantation related disease i.e. graft rejection disease.

Specific examples of transplantation-related diseases which may be treated according to the teachings of the present invention include but are not limited to host vs. graft disease, chronic graft rejection, subacute graft rejection, hyperacute graft rejection, acute graft rejection, allograft rejection, xenograft rejection and graft-versus-host disease (GVHD).

According to specific embodiments, the disease is chronic inflammation.

Specific examples of chronic inflammation which may be treated according to the teachings of the present invention include but are not limited to ileitis (e.g. Crohn's disease), inflammatory bowel disease (IBD, e.g. colitis, ulcerative colitis), chronic viral infection, end-stage heart disease, end-stage renal disease, chronic obstructive pulmonary disease, muscle wasting diseases associated with chronic inflammation (e.g., skeletal muscle loss resulting from age-associated wasting, wasting associated with long-term hospitalization, wasting associated with muscle disuse, wasting associated with muscle immobilization, and wasting associated with chemotherapy or long-term steroid use), cachexia due to cancer and human immunodeficiency virus/acquired immune deficiency syndrome (HIV/AIDS).

According to specific embodiments, the disease is cancer.

Cancers which can be treated by the method of this aspect of some embodiments of the invention can be any solid or non-solid cancer and/or cancer metastasis. Specific examples of cancer which may be treated according to the teachings of the present invention include but are not limited to carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amenable for treatment of the invention include metastatic cancers.

Any of the above agents of the invention can be administered to an organism per se, or in a pharmaceutical composition where it is mixed with suitable carriers or excipients.

Thus, according to another aspect of the present invention there is provided a pharmaceutical composition comprising, as an active ingredient, an agent which inhibits Gads (SEQ ID NO: 1) dimerization as disclosed herein; and a pharmaceutically acceptable carrier or excipient.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent accountable for the biological effect, i.e. inhibiting Gads dimerization.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide.

In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., allergy, autoimmune disease, chronic inflammation, cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in-vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in-vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to specific embodiments, the agent of the present invention can be used alone or in combination with other established or experimental therapeutic regimen to treat e.g. allergy, autoimmune disease, chronic inflammation, cancer.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Antibodies—

The monoclonal antibody C305 (57) was used for anti-TCR stimulations of Jurkat-derived cell lines. Other antibodies used were: anti-human CD69-PE/Cy5, anti-CD16/32, anti-mouse CD63-PE, anti-mouse CD107a-APC and the isotype control Rat IgG2b-APC (all from Biolegend); Rabbit anti-Gads and rabbit anti-PLC-γ1 (Santa Cruz Biotechnology); anti-phospho-LAT (pY132, from Biosource); anti-phospho-PLC-γ1 (pY783, MBL International); rabbit anti-GFP (a gift from Ariel Stanhill); IgE (anti-DNP, Sigma-Aldrich); anti-IgE-PE (Southern Biotech); anti-mouse CD117 (cKit)-APC (Biogems); and anti-mouse IL-6-PerCP-eflour710 (eBioscience).

Plasmids—

For expression of recombinant maltose-binding protein (MBP)-tagged Gads, the open reading frame of human Gads cDNA (NM_001291825.1, SEQ ID NO: 44) was cloned into the BamHI and EcoRI sites of pMAL-C5x (NEB). Deletion mutants were derived by PCR amplification of the entire plasmid, using Phusion Hot Start Flex DNA polymerase (NEB), and 5′ phosphorylated primers that border the deleted area on both sides. Resulting PCR products were circularized with Fast link Ligase (Epicentre). Mutant MBP-Gads constructs had deletion of the following residues: ΔN-SH3 Gads—Δ2-53; ΔCSH3 Gads—Δ274-328; Δlinker Gads—Δ154-267; ΔC-SH3+linker—Δ154-328; SH2 only—Δ2-53 & Δ154-330. His-tagged constructs were created by replacing the MBP reading frame with an N-terminal 6-His tag. The SH2 only construct encoded residues Q54-Q153 of Gads, and was tagged at the N-terminus with either MBP or His (his-SH2 is set forth in SEQ ID NO: 45).

For retroviral infections, full length Gads open reading frame (NM_001291825.1, SEQ ID NO: 44), with an N-terminal twin-strep tag (54) was subcloned into the pMIGR vector, which contains an IRES-GFP cassette (55). The A206K GFP mutation was incorporated to prevent GFP dimerization (56) and a phusion-based strategy was used to remove the IRES sequence and fuse the C-terminus of Gads with monomeric GFP, creating one open reading frame encoding twin-strep-Gads-monomeric GFP. Point mutations in Gads were created by quikchange. All constructs were verified by sequencing the entire open reading frame, using standard Sanger sequencing (BigDye Terminator v1.1 Cycle Sequencing Kit) analyzed by the 3500xL Genetic Analyzer instrument.

Production and Purification of Recombinant Proteins—

E. coli strain BL21 codon-plus (Agilent technologies), expressing MBP- or His-tagged Gads was grown in autoinduction medium (58), containing 5 μg/ml carbenicillin and 25 μg/ml chloramphenicol for 4 hours at 37° C., followed by 16 hours at 18° C. Cells were harvested by centrifugation at 8000 g for 50 minutes at 4° C., and resuspended in column buffer (20 mM HEPES pH 7.3, 100 mM NaCl, 1 mM EDTA, 10% glycerol) for MBP-tagged proteins or binding buffer (20 mM HEPES pH 7.3, 200 mM NaCl, 20 mM Imidazole) for His-tagged proteins, containing protease inhibitors and DNase. Following, cell were disrupted by EmulsiFlex-C3 (Avestin). All purification steps were conducted at 4° C. Lysates were centrifuged at 10,000 g for 50 min, and the supernatant was applied onto a pre-equilibrated, 2.5 cm diameter, 4 ml bed volume gravity column (Econo-Column, Bio-Rad). MBP proteins were incubated with amylose resin (Biolabs) for 2 hours, washed three times and eluted for 2 hours in column buffer supplemented with 10 mM maltose. His-tagged proteins were incubated with Ni-NTA His-Bind Resin (Qiagen) for 2 hours, washed with binding buffer and eluted in binding buffer containing 300 mM imidazole. The eluted proteins were collected and concentrated using Amicon™ Ultra-15 Centrifugal Filter Unit (MBP—30,000 MWCO, His—3000 MWCO).

Size-Exclusion Chromatography and Multi-Angle Light Scattering (SEC-MALS)—

The average molecular weight of Gads proteins was determined by SEC-MALS. The system consisted of an ÄKTA avant 25, coupled to a UV detector (GE Healthcare) and a miniDAWN triple-angle light-scattering detector (Wyatt Technology). 300 μg of MBP Gads protein, either full length or SH2 alone was loaded into a Superdex 200 10/300 column (GE Healthcare) and eluted at 0.5 ml/min with column buffer (20 mM HEPES pH 7.3, 100 mM NaCl, 1 mM EDTA, 10% glycerol). Data collection and analysis was performed with Wyatt's ASTRA 6.1.1 software.

Fast Protein Liquid Chromatography (FPLC)—

MBP- and His-tagged proteins were resolved by size exclusion chromatography at 12° C., using an ÄKTA FPLC system (GE Healthcare), fitted with Superdex 200 10/300 or 16/60 HiLoad for MBP-tagged proteins or Superdex 75 10/300 for His-tagged proteins, in column buffer containing 20 mM HEPES pH 7.3, 100 mM NaCl, 1 mM EDTA, 10% glycerol.

Isothermal Titration Calorimetry (ITC)—

ITC was carried out at 25° C. on a MicroCal 200 titration microcalorimeter (GE Healthcare) with all components resuspended or purified in column buffer (20 mM HEPES pH 7.3, 100 mM NaCl, 1 mM EDTA, 10% glycerol). In brief, 2 μL aliquots of 0.2 mM peptide solution were injected from a rotating syringe at 800 rpm into a sample cell containing 234 μL of 0.02 mM MBP-Gads SH2 solution. The duration of each injection was of 4 s, while the delay between injections was 180 s. Data analysis was performed using Affinimeter.

Peptides—

LAT peptides were synthesized and purified by GL Biochem (Shangai) or Pepmic Co., Ltd (Suzhou) and were validated by mass spectrometry and HPLC. Peptides were 29 residues long: DDYVNVPESGESAEASLDGSREYVNVSQE (SEQ ID NO: 46), encompassing LAT tyrosines 171 and 191; and either doubly phosphorylated (2pY-LAT, DDpYVNVPESGESAEASLDGSREpYVNVSQE, SEQ ID NO: 32) or singly phosphorylated on Y171 (pY171-LAT, DDpYVNVPESGESAEASLDGSREYVNVSQE, SEQ ID NO: 31). Peptides were resuspended in column buffer (0.02 M HEPES pH 7.3, 0.1 M NaCl, 0.01 M EDTA, 10% glycerol). To determine the precise concentration of pY171-LAT peptide, it's binding to purified MBP-Gads SH2 domain was measured by ITC; and the peptide concentration was adjusted to reflect a 1:1 stoichiometry. Concentration of 2pY-LAT was also determined by ITC, by measuring its ability to sequester MBP-Gads SH2 molecules and prevent their binding to pY171-LAT.

Thermal Stability Measurement—

Nano-Differential Scanning Fluorimetry (Nano-DSF) was performed using the Prometheus NT.48 instrument (NanoTemper Technologies, Munich, Germany) to detect the shift in intrinsic tryptophan fluorescence that occurs upon protein denaturation (35). This instrument is also equipped with a light-scattering detector to measure the onset of protein aggregation. 20 μM of purified recombinant Gads protein was loaded into nano-DSF-grade standard capillaries and the temperature was increased at a rate of 1° C./min from 15 to 95° C. while measuring the ratio of tryptophan fluorescence emission intensity (FI350/330). The melting (thermal unfolding) temperature (Tₘ) and onset of aggregation were determined as described (35), using Nanotemper software.

Ras Recruitment System—

Yeast growth, transfection and functional screening for bait-prey interaction using the Ras Recruitment System (RRS) were conducted as described in (49). Gads RRS bait was cloned into p-Met-myc-Ras (49) with full-length Gads fused in frame C-terminal to the Ras protein (SEQ ID NO: 47); and Gads prey was designed by cloning full length Gads into pMyr (50) in frame with an N-terminal myristoylation sequence (SEQ ID NO: 48). The Gads ΔN RRS bait was similar to the full length Gads bait, except for deletion of the sequences coding for Gads amino acids 2-53 (SEQ ID NO: 49). CDC25-2, temperature-sensitive yeast cells were transfected with the indicated plasmids: Ras-Bait and Myristoylated-Prey. Transformants were selected at the permissive temperature (25° C.) and subsequently replica plated onto appropriate medium and grown at the restrictive temperature (36° C.). In this system, growth at 36° C. indicates an interaction between the bait and prey proteins.

Computational Analysis of the SH2 Dimer Interface—

The Gads SH2 dimer interface was determined by visual inspection of adjacent SH2 units in the crystal structure of murine Gads SH2 bound to a short LAT pY171 fragment (PDB1RIP). The structure was also analyzed using pdbsum, which can be found at: www(dot)ebi(dot)ac(dot)uk/thornton-srv/databases/cgi-bin/pdbsum/GetPage(dot)pl?pdbcode=index(dot)html.

To analyse evolutionary conservation of Gads SH2 and LAT, the NCBI blastp (protein-protein blast) program was used to identify orthologs; and 15 mammalian orthologs were aligned using EMBL-EBI Clustal Omega (www(dot)ebi(dot)ac(dot)uk/Tools/msa/clustalo/).

Cell Lines—

The Gads-deficient Jurkat-derived T cell line, dG32, was previously described (21). dG32 cells were retrovirally reconstituted with N-terminally twin-strep-tagged Gads-GFP, followed by FACS sorting for cells with similar expression of GFP.

Affinity Purification and Western Blotting—

Jurkat and dG32-derived T cell lines were stimulated for one minute at 37° C. with anti-TCR (C305). Following, cells were lysed at 10⁸ cells/ml in ice-cold lysis buffer, containing 20 mM Hepes pH 7.3, 1% Triton X-100, 150 mM NaCl, 10% glycerol, 10 mM NaF, 1 mM Na₃VO₄, 10 μg/ml aprotinin, 2 mM EGTA, 10 μg/ml leupeptin, 2 mM phenylmethanesulfonyl fluoride, 1 μg/ml pepstatin and 1 mM dithiothreitol, as described (14). For immunoprecipitation experiments, lysis buffer was supplemented with 0.1% n-Dodecyl-β-D-maltoside (Calbiochem). Lysates were centrifuged twice at 16,000 g for 10 minutes at 4° C.; and twin-strep tagged Gads-GFP was affinity purified by tumbling end over end for 30 minutes at 4° C. with Strep-Tactin Superflow high capacity beads (IBA), using approximately 7 μl bead suspension for every 20 million cells lysed. Following three rapid washes with cold lysis buffer, the isolated complexes were analyzed by western blotting.

Barcoding—

To decrease experimental variation, a barcoding approach was adapted (51), in which cell lines were differentially labeled with four-fold dilutions of CellTrace Violet or CellTrace Far Red cell (Life Technologies). For labeling, PBS-washed cells were incubated with gentle mixing for 20 minutes in the dark at RT, in 0.015-3.75 μM of CellTrace Violet or 0.003-0.75 μM of CellTrace Far Red in PBS. Staining was stopped by adding 4 volumes of medium containing 10% FCS, and incubating for an additional 5 minutes at RT. Cells were then washed and mixed together in the same tube, prior to stimulation and FACS-based functional assays. Data analysis was performed while gating on the differentially barcoded populations within the sample. In all cases, controls were performed to verify that the barcoding reaction had no effect on cellular responsiveness in the assay.

TCR-Induced CD69 Expression—

was measured essentially as described in (21), except that prior to stimulation cells were barcoded with CellTrace Violet stain. Median TCR-induced CD69 expression was normalized to the median PMA-induced expression within the same cell population gate.

Mice—

Wild-type Balb/c mice (WT, from Harlan) and Gads-deficient mice (28) on Balb/c genetic background (59) were used. Mice were maintained under specific pathogen-free conditions, under veterinary supervision, in accordance with the guidelines of the institutional animal ethics committee.

Generation of BMMCs—

Bone marrow cells were obtained from femurs and tibias of WT or Gads-deficient mice and cultured in mast cell medium (Iscove's Modified Dulbecco's, supplemented with 16% iron fortified-bovine calf serum, 100 U/ml penicillin, 100 μg/ml streptomycin, 2 mg/ml glutamine, 50 μM 2-mercaptoethanol, 1 mM sodium pyruvate, 1× nonessential amino acids, 10 mM HEPES) containing 10 ng/ml interleukin 3 (IL-3, PeproTech) and 10 ng/ml stem cell factor (SCF, PeproTech). Retroviruses encoding different alleles of Gads-GFP, or GFP alone were packaged in Plat E cells (Cell Biolabs), using lipofectamine 3000 transfection reagent (Invitrogen). Cells were infected twice, on day two and three of culturing, and were sorted for GFP⁺ cells during the fourth week. Experiments started once the cells were ≥95% cKit⁺, FcεRI⁺, as shown by FACS staining.

FcεRI Signaling Assays—

Fully differentiated mast cells were washed in cytokine-free medium, barcoded, and sensitized overnight at 37° C. in medium containing 10 ng/ml IL-3 and 0.1 μg/ml IgE (anti-DNP). For Ca²⁺ assays, CellTrace Far Red-barcoded, sensitized BMMCs were washed and incubated for 20 minutes at 37° C. in Tyrode's buffer (52) containing 1 mM Probenicid (Sigma-Aldrich) and 3 μg/ml Indo-1-AM (eBioscience), diluted 10-fold in 37° C. Tyrode's buffer for an additional 20 minutes, then washed twice and resuspended at 2×10⁶ cells/ml in Tyrode's buffer. Intracellular calcium was measured ratiometrically by flow cytometry at 37° C., with the indicated concentration of dinitrophenol-conjugated human serum albumin (DNP-HSA; Sigma) added at the 60 sec time point. CellTrace Violet-barcoded, pre-sensitized BMMCs were used to assess degranulation and IL-6 production. For degranulation, cells were stimulated with DNP-HSA for 15 minutes in Tyrode's buffer, followed by fixation with 2% PFA for 15 minutes at RT; and staining with PE conjugated anti-CD63 or APC-conjugated anti-CD107a. IL-6 production was assessed following 4.5 hours of stimulation with DNP-HSA by intracellular staining with IL-6-PerCP-eflour710, as described in (41).

### Example 1

### Spontaneous Dimerization of Gads Via its SH2 Domain

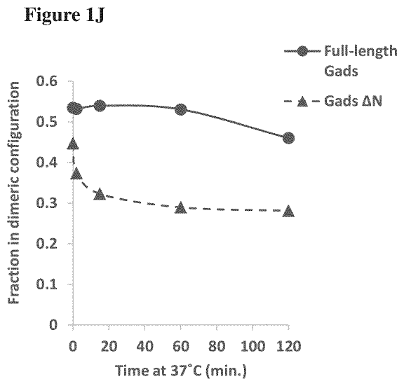

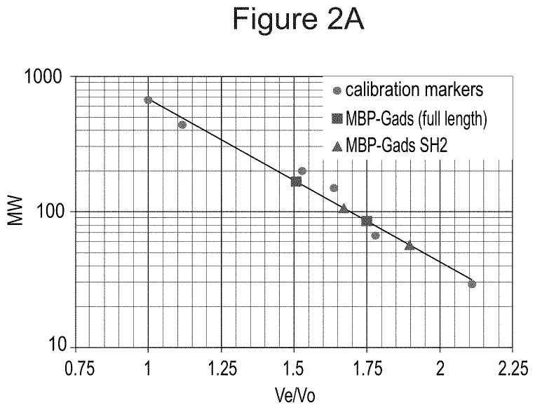

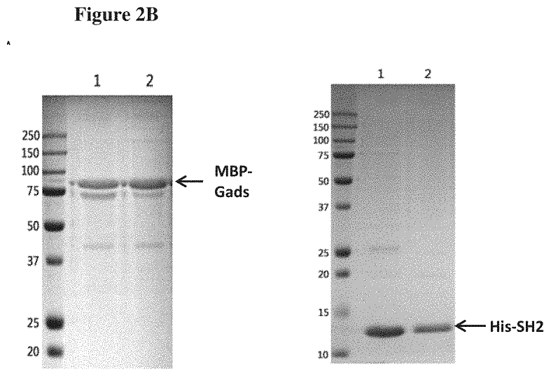

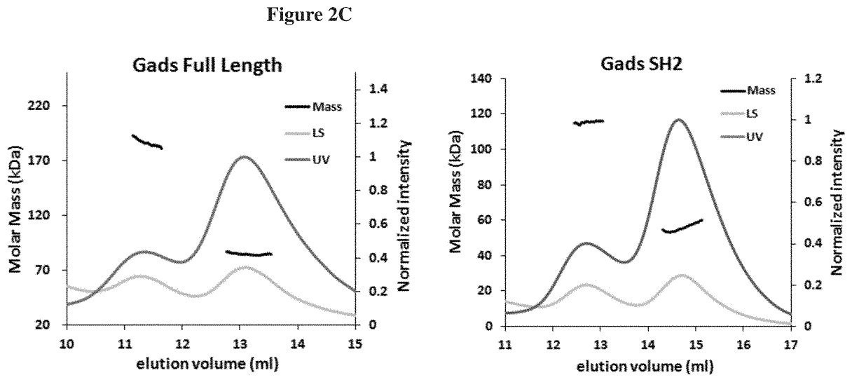

Gads contains a single SH2 domain, yet requires two LAT pTyr sites for efficient binding (8). To explore this discrepancy, recombinant MBP-Gads was resolved by size exclusion chromatography, which separates proteins based on their globular radius. As shown in FIG. 1A, full length Gads resolved into two main peaks, with elution volumes corresponding to the predicted molecular weight of monomeric and dimeric MBP-Gads (FIG. 2A). SDS-PAGE analysis confirmed that both peaks contained an identical protein species, at the expected molecular weight of MBP-Gads (FIG. 2B, left). To rule out partial protein unfolding as the source of either peak, the protein denaturation temperature (Tₘ) was measured by nano-DSF, a technique in which proteins are gradually heated, while measuring the shift in intrinsic tryptophan fluorescence that occurs upon their unfolding (35). Both peaks exhibited Tm in the range of 56.5-56.7° C. (FIG. 1B), suggesting that they represent alternative, stably folded conformations of Gads-MBP protein. Size-exclusion chromatography and multi-angle light scattering (SEC-MALS) analysis confirmed that the earlier-eluting peak has twice the molecular weight of the later peak (FIG. 2C), suggesting that it represents a spontaneously dimerized form of Gads.

Gads N- and C-terminal SH3 domains and linker region were not required for its resolution into two peaks, indicated by the fact that MBP-Gads proteins, either wild-type or lacking the N-SH3 domain, the C-SH3 domain and/or the linker domain (FIG. 1C) were all resolved into two peaks (data not shown). Indeed, purified MBP-SH2 resolved to two peaks at the expected size of its monomeric and dimeric forms (FIG. 2A). The MBP tag facilitated the purification and storage of recombinant Gads proteins, but was not required for dimerization, as His-tagged Gads SH2 resolved by size exclusion chromatography into two peaks (FIG. 1D) that exhibited identical mobility by SDS-PAGE (FIG. 2B, right), as well. These results establish spontaneous dimerization as an intrinsic property of the Gads SH2 and show that the Gads SH2 domain alone is sufficient for dimerization.

To assess the stability of spontaneous Gads dimerization, MBP-Gads proteins from the dimeric fraction were stored on ice or incubated at 37° C.; and the resulting oligomerization state was determined by size exclusion chromatography. Full length Gads protein from the dimeric fraction re-equilibrated on ice to a mixture of monomeric and dimeric forms, with the equilibrium shifting slightly towards the monomeric form at 37° C. (FIG. 1E, left). A substantial fraction remained dimeric even following 2.5 hours at 37° C., suggesting that Gads spontaneous dimerization is relatively stable at physiologic temperature. In contrast, the isolated SH2 domain converted rapidly to the monomeric form at 37° C. (FIG. 1E, right), suggesting that additional Gads domains are required to stabilize the dimeric conformation at physiologic temperature. Gads lacking the N-SH3 produced spontaneous dimers that were less stable at 37° C. than those formed by full length Gads, but more stable than the dimers formed by the SH2 alone; demonstrating that the N-SH3 makes a measurable contribution to stabilizing the dimeric state (FIGS. 1E and 1I-J).

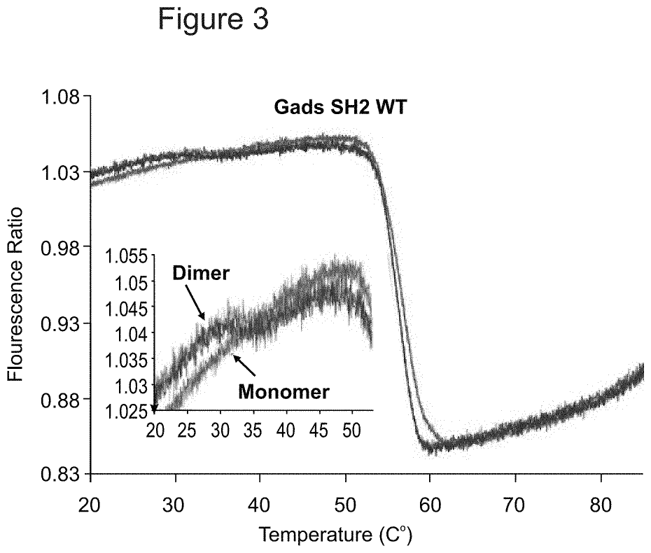

The relative instability of dimerization of the isolated Gads SH2 was further supported by nano-DSF analysis, which detects the increased solvent exposure of tryptophan residues, resulting in a shift of intrinsic fluorescence as proteins unfold (35). It was considered that the two tryptophan residues found in Gads SH2 may be shielded from the solvent by dimerization, such that their intrinsic fluorescence may be affected by the SH2 dimerization state. Consistent with this, nano-DSF analysis of dimeric, His-tagged Gads SH2 revealed two temperature-dependent transitions. The first occurred at 32.5° C. and was not associated with protein aggregation, suggesting that it represents the temperature of monomerization. A second transition, in the range of 54-57° C., was observed for both monomeric and dimeric Gads SH2 and was accompanied by protein aggregation, suggesting that it represents the Tm (FIG. 1F). A similar low-temperature transition was observed in the nano-DSF profile of dimeric MBP-Gads SH2 (FIG. 3); this transition was subtle, due to the presence of 8 additional tryptophan residues in the MBP tag that are not affected by SH2 dimerization. Taken together, these data support the notion that the earlier-eluting Gads SH2 peak represents a well-folded, spontaneously dimerizing form, which dissociates to a well-folded monomeric form at approximately 32° C.

To confirm self-association of full length Gads at physiologic temperature in intact cells, the Ras-Recruitment System (RRS) was used. RRS is a type of yeast two-hybrid system, in which the interaction of bait and prey proteins is required for yeast growth at the restrictive temperature (36). Growth was clearly observed when full length Gads was used both as bait and prey (FIG. 1G, row 2), but no growth occurred when either the bait or the prey was eliminated (FIG. 1G, rows 1 and 4). Since this assay is performed at 36° C., it provides good evidence that self-association of full length Gads can occur at physiologic temperature and regulate its signaling function within cells. In addition, when the bait protein was lacking the N-terminal SH3 (N-SH3) of Gads, Gads self-association was abolished (FIG. 1H), suggesting that SH2 dimerization is stabilized by additional interactions mediated by the N-SH3 domain. A homology modeling program was used to create a predicted structure of the SH3 domain of Gads. This prediction demonstrated that the N-SH3 is expected to have a large hydrophobic surface, which is not typical of a cytosolic protein. It is postulated that the hydrophobic surface of the N-SH3 is buried, either by self-association of the N-SH3 (which would stabilize the dimeric form of Gads); or by associating with other hydrophobic surfaces found in Gads (e.g. on the C-terminal SH3 domain).

### Example 2

### Identification of the Gads SH2 Dimerization Interface

While examining a previously determined structure of murine Gads SH2 co-crystallized with a short phospho-LAT peptide (PDB 1R1P, 37), the present inventors first identified that the minimal asymmetric unit included two pairs of closely associated Gads SH2 domains, each bound to a phospho-LAT peptide (FIG. 4A, left). Within each pair, two SH2 domains appear to be held together by hydrophobic interactions between F92 on adjacent domains as well as hydrogen bonds between R109 on one partner and D91 on the other (FIG. 4A, right). A space filling model more clearly demonstrated the tight association at the putative SH2 dimerization interface which features an area of approximately 850 A² (FIG. 5A), a value that falls within the range of known dimerization interfaces (38).

Visual inspection of the structure revealed 24 residues within the dimer interface (FIGS. 5B-C), 14 of them were determined to be core residues. Multiple sequence alignment of Gads SH2 domain from 15 mammalian species (FIG. 5D) indicated that the residues found in the dimerization interface were highly conserved between the species. Additionally, these residues are relatively unique to Gads as they are not conserved between different SH2 domains.

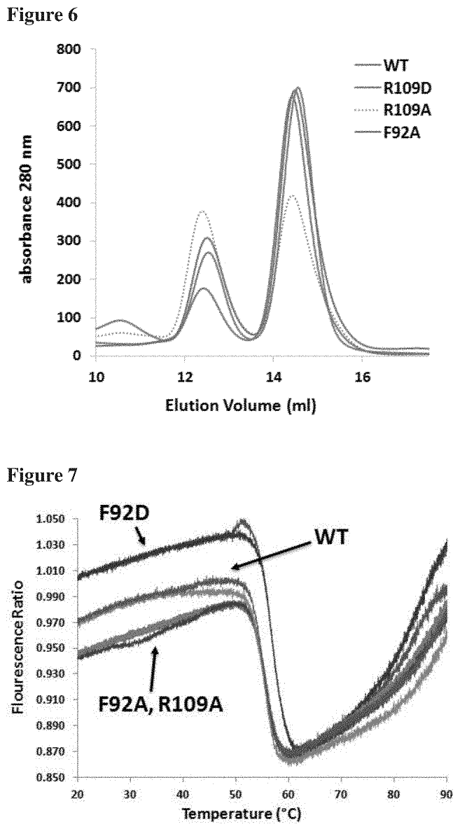

To disrupt the dimerization interface, F92 and R109 which are located at the center of the dimerization interface (FIG. 5F) were mutated to alanine, in the context of MBP-Gads SH2. Neither mutation alone was sufficient to disrupt dimerization (FIG. 6), but the F92A/R109A double mutation completely disrupted spontaneous Gads SH2 dimerization (FIG. 4B). The present inventors reasoned that a more dramatic effect might be obtained by mutating these residues to a negative charge. To this end, mutation of R109 to D was insufficient to disrupt dimerization (FIG. 6); however, the F92D mutation completely abolished spontaneous Gads SH2 dimerization (FIG. 4B).

Despite their profound effect on Gads dimerization, the F92D and F92A/R109A mutations only moderately reduced the affinity with which Gads SH2 bound to a mono-phosphorylated, LAT pY171 peptide (FIG. 4D). Wild-type Gads SH2 bound pY171-LAT with a K_{D} (1/K_{A}) of 177 nM, within the range of previously reported values (25, 37). Mutational inactivation of the dimerization interface moderately increased the K_{D} to 470 nM for the F92D SH2 and 335 nM for F92A/R109A SH2 mutant. These results are consistent with proper folding of the SH2 domain, and suggest that the Gads SH2 dimerization interface is largely distinct from the pTyr-binding pocket.

In the context of full length Gads, both the F92D single mutation and the F92A/R109A double mutation abolished Gads dimerization (FIG. 4C), without adversely affecting the stability of protein folding (FIG. 7).

Taken together, the data identify an SH2 interface comprising F55, P56, W58, F59, E61, G62, A84-F92, V107-N111 (in human Gads, corresponding to V107-T11 in murine Gads), Y115, F116, L125 and N126 that is required for dimerization of Gads.

### Example 3

### Gads Dimerization Facilitates its Cooperative Binding to Dual-Phosphorylated LAT

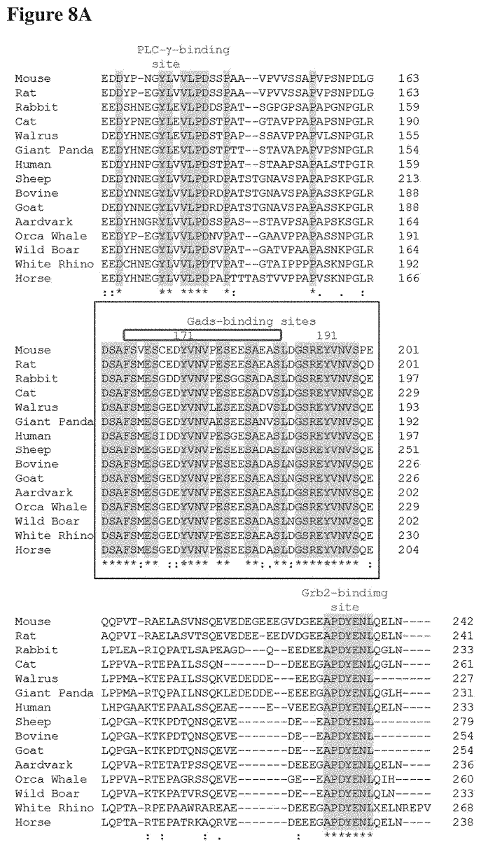

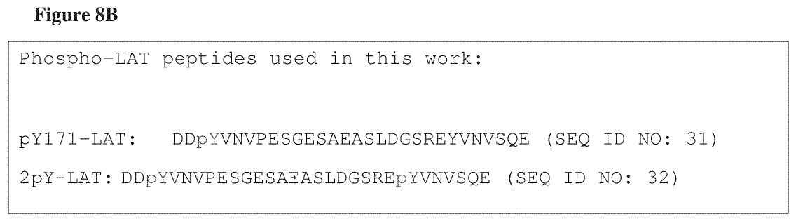

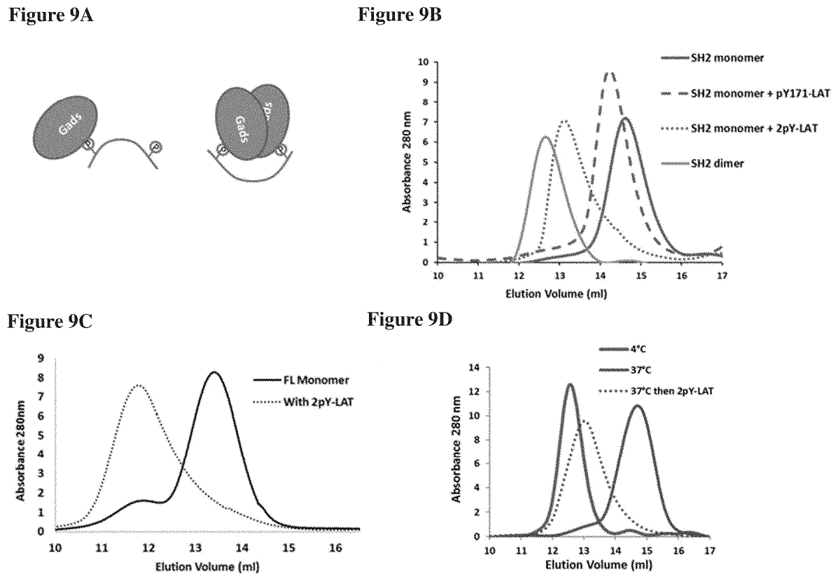

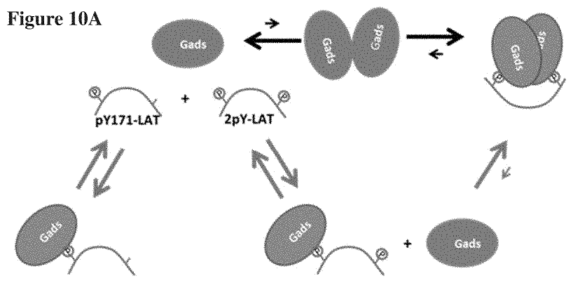

Gads binds LAT at pY171 and pY191, sites that are found at an evolutionarily conserved distance from each other, connected by a highly conserved linker sequence (boxed region, FIG. 8A), suggesting that they may function as a unit. To test whether transient Gads dimerization could facilitate its cooperative binding to pY171 and pY191 on LAT (8), monomeric Gads SH2 was incubated with a molar excess of synthetic LAT peptide, encompassing both Gads-binding sites, and phosphorylated at both (2pY-LAT, SEQ ID NO: 32) or one (pY171-LAT, SEQ ID NO: 31). Two possible modes of Gads binding to 2pY-LAT were envisioned, single and paired (FIG. 9A). To ensure the availability of both modes, a 7-fold molar excess of 2pY-LAT peptide was applied. In the absence of cooperativity, the large molar excess of unbound SH2-binding sites should favor unpaired binding (FIG. 9A, left). Binding of monomeric Gads SH2 to pY171-LAT resulted in a small Gads mobility shift, consistent with the added weight of the bound peptide (FIG. 9B, solid and dashed red lines). In contrast, 2pY-LAT induced a dramatic shift towards the dimeric form, indicating that paired binding was favored (FIG. 9B, red dotted line). 2pY-LAT induced a similar shift of monomeric full length Gads to the dimeric form, indicating preferentially paired binding of full length Gads to LAT (FIG. 9C).

The 2pY-LAT-induced SH2 dimer is more compact than the spontaneous dimer, as evident by its later elution on size exclusion chromatography (FIG. 9B). Moreover, LAT-induced SH2 dimers exhibited increased stability at 37° C. To demonstrate this difference, spontaneous Gads SH2 dimers were briefly incubated at 37° C., inducing their conversion to the monomer form; and upon subsequent addition of 2pY-LAT peptide at 37° C., dimerization was induced (FIG. 9D).

These results suggest that binding to 2pY-LAT markedly stabilizes the dimeric form of Gads.

To test whether Gads dimerization promotes preferential binding to dual-phosphorylated LAT molecules, competitive binding experiments were performed, in which monomeric Gads SH2 was incubated with a mixture of 2pY-LAT and pY171-LAT peptides (FIG. 10A). The proportion of Gads bound to each peptide was distinguished by their mobility on size exclusion chromatography.

As shown in FIG. 10B (left histogram), pY171-LAT (SEQ ID NO: 31) competitor peptide shifted the SH2 binding equilibrium somewhat towards the monomeric mode; nevertheless, over two thirds of wild-type Gads SH2 remained in the paired binding mode, even when pY171-LAT (SEQ ID NO: 31) was present at twice the concentration of 2pY-LAT (SEQ ID NO: 32). This type of preferentially paired binding to dual-phosphorylated LAT was observed over a wide range of pY171-LAT competitor concentrations (FIG. 10C, blue curve), with over half of Gads SH2 molecules exhibiting paired binding, even at a four-fold excess of competitor peptide. Similar results were observed for full length, wild-type Gads (FIG. 10D, left histogram).

Together, these results indicate an intrinsic ability of Gads SH2 to discriminate between singly and doubly phosphorylated LAT molecules, by preferentially binding to the latter.

Compared to wild type Gads SH2, F92D SH2 exhibited a reduced preference for paired binding (FIG. 10B); as lower concentrations of competitor peptide sufficed to inhibit 2pY-LAT-induced dimerization (FIG. 10C). Moreover, full length Gads F92D, and F92A/R109A proteins exhibited profound impairment of 2pY-LAT-induced dimerization, as well as impaired discrimination between dual- and single-phosphorylated LAT (FIG. 10D). Together, these results suggest that the selectivity of Gads SH2 for dual-phosphorylated LAT depends on its dimerization interface, which is likewise required to support paired binding of full length Gads to LAT.

Preferentially paired binding of Gads SH2 to LAT suggests positive cooperativity, which may result from increased affinity of Gads for a second binding site, once the first site is bound (39). Alternatively, cooperativity may reflect the formation of a multimolecular complex (39), in which transient Gads dimers bind LAT at an overall affinity that is higher than the product of the individual site-specific binding constants (40).

### Example 4

### Gads-Dependent TCR Signaling Depends on an Intact Gads Dimerization Interface

To explore the importance of Gads dimerization in intact T cells, dG32, a Jurkat-derived Gads-deficient T cell line (21) was reconstituted with N-terminally twin-strep-tagged, full length human Gads-GFP, either wild type (WT), F92D or F92A/R109A; and a wide range of GFP⁺ cells were isolated by sorting (FIG. 11A, left panel). TCR-induced CD69 expression increased with increasing expression of WT Gads as expected (21), but not in cells expressing Gads F92D or Gads F92A/R109A, where it remained at the level observed in Gads-deficient cells (FIG. 11A).

In the next step, Gads-GFP-reconstituted cells were sorted for equal and homogeneous GFP expression, stimulated via the TCR; and the molecular interactions and downstream signaling events mediated by Gads were assessed. Most strikingly, mutation of the Gads SH2 dimerization interface abolished the TCR-induced recruitment of Gads to phospho-LAT (FIG. 11B). This effect was specific, as neither LAT phosphorylation nor Gads interaction with SLP-76 were affected (FIG. 11B). Consistent with the impaired recruitment of Gads to LAT, TCR-induced phosphorylation of PLC-γ1 was markedly impaired in F92D- and F92A/R109A-reconstituted cells, which resembled Gads-deficient cells (FIG. 11C).

The strikingly defective LAT-binding of the dimerization-defective Gads mutants is consistent with cooperatively paired binding of Gads to LAT and suggests that cooperative, preferentially paired binding of full length Gads to dual-phosphorylated LAT molecules is required to stabilize LAT complex formation.

Taken together, these results demonstrate that Gads SH2 dimerization interface is specifically required to support its signaling functions in the TCR pathway.

### Example 5

### Gads-Dependent FcεRI Signaling Depends on an Intact Gads Dimerization Interface

To assess the importance of Gads dimerization in FcεRI signaling, Gads-deficient murine bone marrow was retrovirally reconstituted with wild-type (WT) or F92D GFP-tagged Gads, followed by in-vitro differentiation to the mast cell lineage. Fully differentiated BMMCs, either WT, Gads-deficient or Gads-reconstituted, were sensitized with DNP-specific IgE, which bound equally to all cell types.

FcεRI signaling was initiated by the addition of DNP-HSA at 37° C. and three different responses were measured, representing different time scales:

1. calcium flux, which occurs immediately following addition of DNP-HSA (FIG. 12A);

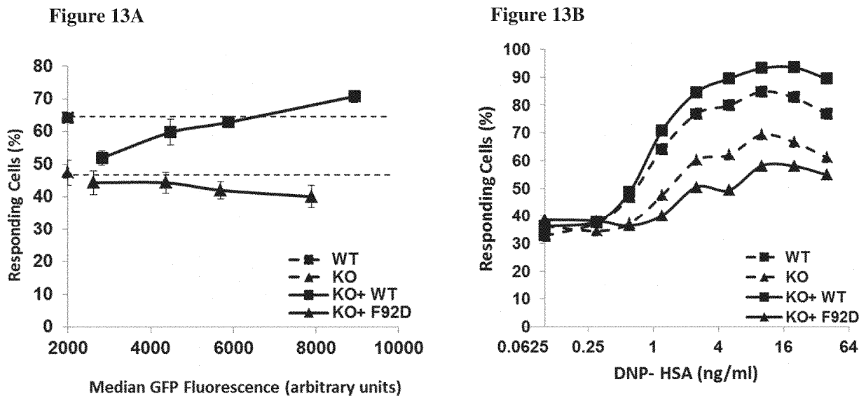

2. degranulation, which occurs in the first 15 minutes following addition of DNP-HSA (FIGS. 12B and 13A-B); and

3. IL-6 cytokine production, which occurs over a few hours following addition of DNP-HSA (FIG. 12C).

FACS-based assays revealed binary BMMCs responses in all three assays, with Gads-deficient cells exhibiting a lower frequency of response (FIGS. 12A-C). The proportion of Gads-deficient BMMCs responding with increased intracellular calcium was most markedly reduced when the intensity of stimulation was low (FIG. 12A). Calcium triggers degranulation, bringing two proteins to the cell surface: CD63 and CD107a (23, 41). Cell surface expression of both markers was reduced in Gads-deficient cells, at all levels of FcεRI simulation (FIG. 12B, right histogram and FIGS. 13A-B). Finally, the proportion of Gads-deficient cells exhibiting FcεRI-induced IL-6 production was reduced at all levels of FcεRI stimulation (FIG. 12C, right).

The response of WT Gads-GFP-reconstituted BMMCs was strongly dependent on the level of Gads expression (FIG. 12B, middle). Therefore, all responses were analyzed while gating on a narrow, equivalent level of expression of WT and mutant Gads-GFP.

WT Gads-GFP-reconstituted BMMCs responded similarly to WT BMMCs in all three assays, whereas F92D-reconstituted cells responded similarly to Gads-deficient BMMCs (FIGS. 12A-C, right panels).

Taken together, these results demonstrate that Gads SH2 dimerization interface is specifically required to support its signaling functions in the FcεRI pathway.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

(other references are cited throughout the application)
1. T. Kambayashi, G. A. Koretzky, Proximal signaling events in FcεRI-mediated mast cell activation. Journal of Allergy and Clinical Immunology 119, 544-552 (2007).
2. R. J. Brownlie, R. Zamoyska, T cell receptor signalling networks: branched, diversified and bounded. Nat Rev Immunol 13, 257-269 (2013).
3. A. K. Chakraborty, A. Weiss, Insights into the initiation of TCR signaling. Nat Immunol 15, 798-807 (2014).
4. J. C. Houtman, R. A. Houghtling, M. Barda-Saad, Y. Toda, L. E. Samelson, Early phosphorylation kinetics of proteins involved in proximal TCR-mediated signaling pathways. J. Immunol. 175, 2449-2458 (2005).
5. J. Lin, A. Weiss, Identification of the minimal tyrosine residues required for linker for activation of T cell function. J. Biol. Chem. 276, 29588-29595 (2001).
6. P. E. Paz, S. Wang, H. Clarke, X. Lu, D. Stokoe, A. Abo, Mapping the Zap-70 phosphorylation sites on LAT (linker for activation of T cells) required for recruitment and activation of signalling proteins in T cells. Biochem J 356, 461-471 (2001).
7. W. Zhang, R. P. Trible, M. Zhu, S. K. Liu, C. J. McGlade, L. E. Samelson, Association of Grb2, Gads and Phospholipase C-g1 with phosphorylated LAT tyrosine residues. J. Biol. Chem. 275, 23355-23361 (2000).
8. M. Zhu, E. Janssen, W. Zhang, Minimal requirement of tyrosine residues of linker for activation of T cells in TCR signaling and thymocyte development. J Immunol 170, 325-333 (2003).
9. I. K. Jang, J. Zhang, H. Gu, Grb2, a simple adapter with complex roles in lymphocyte development, function, and signaling. Immunol Rev 232, 150-159 (2009).
10. S. K. Liu, D. M. Berry, C. J. McGlade, The role of Gads in hematopoietic cell signalling. Oncogene 20, 6284-6290 (2001).
11. D. M. Berry, P. Nash, S. K. Liu, T. Pawson, C. J. McGlade, A high-affinity Arg-X-X-Lys SH3 binding motif confers specificity for the interaction between Gads and SLP-76 in T cell signaling. Curr Biol 12, 1336-1341 (2002).
12. M. Harkiolaki, M. Lewitzky, R. J. Gilbert, E. Y. Jones, R. P. Bourette, G. Mouchiroud, H. Sondermann, I. Moarefi, S. M. Feller, Structural basis for SH3 domain-mediated high-affinity binding between Mona/Gads and SLP-76. Embo J 22, 2571-2582 (2003).
13. Q. Liu, D. Berry, P. Nash, T. Pawson, C. J. McGlade, S. S. Li, Structural basis for specific binding of the Gads SH3 domain to an RxxK motif-containing SLP-76 peptide: a novel mode of peptide recognition. Mol Cell 11, 471-481 (2003).
14. Y. Bogin, C. Ainey, D. Beach, D. Yablonski, SLP-76 mediates and maintains activation of the Tec family kinase ITK via the T cell antigen receptor-induced association between SLP-76 and ITK. Proc. Natl. Acad. Sci. USA 104, 6638-6643 (2007).
15. S. C. Bunnell, M. Diehn, M. B. Yaffe, P. R. Findell, L. C. Cantley, L. J. Berg, Biochemical interactions integrating Itk with the T Cell Receptor-initiated signaling cascade. J. Biol. Chem. 275, 2219-2230 (2000).
16. M. Sela, Y. Bogin, D. Beach, T. Oellerich, J. Lehne, J. E. Smith-Garvin, M. Okumura, E. Starosvetsky, R. Kosoff, E. Libman, G. Koretzky, T. Kambayashi, H. Urlaub, J. Wienands, J. Chernoff, D. Yablonski, Sequential phosphorylation of SLP-76 at tyrosine 173 is required for activation of T and mast cells. EMBO J 30, 3160-3172 (2011).
17. S. Devkota, R. E. Joseph, L. Min, D. Bruce Fulton, A. H. Andreotti, Scaffold Protein SLP-76 Primes PLCγ1 for Activation by ITK-Mediated Phosphorylation. Journal of Molecular Biology 427, 2734-2747 (2015).
18. H. Asada, N. Ishii, Y. Sasaki, K. Endo, H. Kasai, N. Tanaka, T. Takeshita, S. Tsuchiya, T. Konno, K. Sugamura, Grf40, a novel Grb2 family member, is involved in T cell signaling through interactions with SLP-76 and LAT. J. Exp. Med 189, 1383-1390 (1999).
19. C.-L. Law, M. K. Ewings, P. M. Chaudhary, S. A. Solow, T. J. Yun, A. J. Marshall, L. Hood, E. A. Clark, GrpL, a Grb2-related adaptor protein, interacts with SLP-76 to regulate Nuclear Factor of Activated T Cell activation. J. Exp. Med 189, 1243-1253 (1999).
20. S. K. Liu, N. Fang, G. A. Koretzky, C. J. McGlade, The hematopoietic-specific adaptor protein Gads functions in T-cell signaling via interactions with the SLP-76 and LAT adaptors. Curr. Biol. 9, 67-75 (1999).
21. J. Lugassy, J. Corso, D. Beach, T. Petrik, T. Oellerich, H. Urlaub, D. Yablonski, Modulation of TCR responsiveness by the Grb2-family adaptor, Gads. Cellular signalling 27, 125-134 (2015).
22. S. Saitoh, R. Arudchandran, T. S. Manetz, W. Zhang, C. L. Sommers, P. E. Love, J. Rivera, L. E. Samelson, LAT is essential for FcεRI-mediated mast cell activation. Immunity 12, 525-535 (2000).
23. S. Yamasaki, M. Takase-Utsugi, E. Ishikawa, M. Sakuma, K. Nishida, T. Saito, O. Kanagawa, Selective impairment of FcepsilonRI-mediated allergic reaction in Gads-deficient mice. Int Immunol 20, 1289-1297 (2008).
24. V. I. Pivniouk, T. R. Martin, J. M. Lu-Kuo, H. R. Katz, H. C. Oettgen, R. S. Geha, SLP-76 deficiency impairs signaling via the high-affinity IgE receptor in mast cells. J. Clin. Invest. 103, 1737-1743 (1999).
25. J. C. Houtman, Y. Higashimoto, N. Dimasi, S. Cho, H. Yamaguchi, B. Bowden, C. Regan, E. L. Malchiodi, R. Mariuzza, P. Schuck, E. Appella, L. E. Samelson, Binding specificity of multiprotein signaling complexes is determined by both cooperative interactions and affinity preferences. Biochemistry 43, 4170-4178 (2004).
26. S. L. Dalheimer, L. Zeng, K. E. Draves, A. Hassaballa, N. N. Jiwa, T. D. Parrish, E. A. Clark, T. M. Yankee, Gads-deficient thymocytes are blocked at the transitional single positive CD4+ stage. Eur J Immunol 39, 1395-1404 (2009).
27. T. M. Yankee, T. J. Yun, K. E. Draves, K. Ganesh, M. J. Bevan, K. Murali-Krishna, E. A. Clark, The Gads (GrpL) adaptor protein regulates T cell homeostasis. J Immunol 173, 1711-1720 (2004).
28. J. Yoder, C. Pham, Y.-M. lizuka, O. Kanagawa, S. K. Liu, J. McGlade, A. M. Cheng, Requirement for the SLP-76 adaptor GADS in T cell development. Science 291, 1987-1991 (2001).
29. J. L. Clements, B. Yang, S. E. Ross-Barta, S. L. Eliason, R. F. Hrstka, R. A. Williamson, G. A. Koretzky, Requirement for the leukocyte-specific adapter protein SLP-76 for normal T cell development. Science 281, 416-419 (1998).
30. V. Pivniouk, E. Tsitsikov, P. Swinton, G. Rathbun, F. W. Alt, R. S. Geha, Impaired viability and profound block in thymocyte development in mice lacking the adaptor protein SLP-76. Cell 94, 229-238 (1998).
31. W. Zhang, C. L. Sommers, D. N. Burshtyn, C. C. Stebbins, J. B. DeJarnette, R. P. Trible, A. Grinberg, H. C. Tsay, H. M. Jacobs, C. M. Kessler, E. O. Long, P. E. Love, L. E. Samelson, Essential role of LAT in T cell development. Immunity 10, 323-332 (1999).
32. T. S. Finco, T. Kadlecek, W. Zhang, L. E. Samelson, A. Weiss, LAT is required for TCR-mediated activation of PLCg1 and the Ras pathway. Immunity 9, 617-626 (1998).
33. D. Yablonski, M. R. Kuhne, T. Kadlecek, A. Weiss, Uncoupling of nonreceptor tyrosine kinases from PLC-g1 in an SLP-76-deficient T cell. Science 281, 413-416 (1998).
34. M. Y. Bilal, E. Y. Zhang, B. Dinkel, D. Hardy, T. M. Yankee, J. C. Houtman, GADS is required for TCR-mediated calcium influx and cytokine release, but not cellular adhesion, in human T cells. Cellular signalling 27, 841-850 (2015).
35. D. B. Temel, P. Landsman, M. L. Brader, in Methods in enzymology, L. F. Andrew, Ed. (Academic Press, 2016), vol. Volume 567, pp. 359-389.
36. Y. C. Broder, S. Katz, A. Aronheim, The ras recruitment system, a novel approach to the study of protein-protein interactions. Curr Biol 8, 1121-1124 (1998).
37. S. Cho, C. A. Velikovsky, C. P. Swaminathan, J. C. Houtman, L. E. Samelson, R. A. Mariuzza, Structural basis for differential recognition of tyrosine-phosphorylated sites in the linker for activation of T cells (LAT) by the adaptor Gads. Embo J 23, 1441-1451 (2004).
38. R. P. Bahadur, P. Chakrabarti, F. Rodier, J. Janin, Dissecting subunit interfaces in homodimeric proteins. Proteins: Structure, Function, and Bioinformatics 53, 708-719 (2003).
39. A. Whitty, Cooperativity and biological complexity. Nat Chem Biol 4, 435-439 (2008).
40. E. Freire, A. Schön, A. Velazquez-Campoy, in Methods in enzymology, J. M. H. Michael L. Johnson, K. A. Gary, Eds. (Academic Press, 2009), vol. Volume 455, pp. 127-155.
41. N. Foger, A. Jenckel, Z. Orinska, K. H. Lee, A. C. Chan, S. Bulfone-Paus, Differential regulation of mast cell degranulation versus cytokine secretion by the actin regulatory proteins Coronin1a and Coronin1b. J Exp Med 208, 1777-1787 (2011).
42. N. Schiering, E. Casale, P. Caccia, P. Giordano, C. Battistini, Dimer Formation through Domain Swapping in the Crystal Structure of the Grb2-SH2-Ac-pYVNV Complex‡. Biochemistry 39, 13376-13382 (2000).
43. S. Frese, W.-D. Schubert, A. C. Findeis, T. Marquardt, Y. S. Roske, T. E. B. Stradal, D. W. Heinz, The Phosphotyrosine Peptide Binding Specificity of Nck1 and Nck2 Src Homology 2 Domains. Journal of Biological Chemistry 281, 18236-18245 (2006).
44. R. E. Joseph, N. D. Ginder, J. A. Hoy, J. C. Nix, D. B. Fulton, R. B. Honzatko, A. H. Andreotti, Structure of the interleukin-2 tyrosine kinase Src homology 2 domain; comparison between X-ray and NMR-derived structures. Acta Crystallographica Section F: Structural Biology and Crystallization Communications 68, 145-153 (2012).
45. A. P. Benfield, B. B. Whiddon, J. H. Clements, S. F. Martin, Structural and energetic aspects of Grb2-SH2 domain-swapping. Archives of Biochemistry and Biophysics 462, 47-53 (2007).
46. Z. Ahmed, Z. Timsah, K. M. Suen, N. P. Cook, G. R. Lee Iv, C.-C. Lin, M. Gagea, A. A. Marti, J. E. Ladbury, Grb2 monomer-dimer equilibrium determines normal versus oncogenic function. Nature communications 6, (2015).
47. H. Liu, Y. R. Thaker, L. Stagg, H. Schneider, J. E. Ladbury, C. E. Rudd, SLP-76 Sterile α Motif (SAM) and Individual H5 α Helix Mediate Oligomer Formation for Microclusters and T-cell Activation. Journal of Biological Chemistry 288, 29539-29549 (2013).
48. N. P. Coussens, R. Hayashi, P. H. Brown, L. Balagopalan, A. Balbo, I. Akpan, J. C. Houtman, V. A. Barr, P. Schuck, E. Appella, L. E. Samelson, Multipoint binding of the SLP-76 SH2 domain to ADAP is critical for oligomerization of SLP-76 signaling complexes in stimulated T cells. Mol Cell Biol 33, 4140-4151 (2013).
49. M. Hubsman, G. Yudkovsky, A. Aronheim, A novel approach for the identification of protein-protein interaction with integral membrane proteins. Nucleic Acids Res 29, E18 (2001).
50. T. Wasserman, K. Katsenelson, S. Daniliuc, T. Hasin, M. Choder, A. Aronheim, A Novel c-Jun N-terminal Kinase (JNK)-binding Protein WDR62 Is Recruited to Stress Granules and Mediates a Nonclassical JNK Activation. Molecular Biology of the Cell 21, 117-130 (2010).
51. P. O. Krutzik, M. R. Clutter, A. Trejo, G. P. Nolan, in Current Protocols in Cytometry. (John Wiley & Sons, Inc., 2011).
52. T. Kambayashi, M. Okumura, R. G. Baker, C. J. Hsu, T. Baumgart, W. Zhang, G. A. Koretzky, Independent and cooperative roles of adaptor molecules in proximal signaling during FcepsilonRI-mediated mast cell activation. Mol. Cell. Biol. 30, 4188-4196 (2010).
53. Saitoh, S.-i., S. Odom, G. Gomez, C. L. Sommers, H. A. Young, J. Rivera, and L. E. Samelson, The Four Distal Tyrosines Are Required for LAT-dependent Signaling in FcεRI-mediated Mast Cell Activation. The Journal of Experimental Medicine 198:831-843 (2003).
54. T. G. M. Schmidt, L. Batz, L. Bonet, U. Carl, G. Holzapfel, K. Kiem, K. Matulewicz, D. Niermeier, I. Schuchardt, K. Stanar, Development of the Twin-Strep-tag® and its application for purification of recombinant proteins from cell culture supernatants. Protein Expression and Purification 92, 54-61 (2013).
55. W. S. Pear, J. P. Miller, L. Xu, J. C. Pui, B. Soffer, R. C. Quackenbush, A. M. Pendergast, R. Bronson, J. C. Aster, M. L. Scott, D. Baltimore, Efficient and rapid induction of a chronic myelogenous leukemia-like myeloproliferative disease in mice receiving P210 bcr/abl-transduced bone marrow. Blood 92, 3780-3792 (1998).
56. R. N. Day, M. W. Davidson, The fluorescent protein palette: tools for cellular imaging. Chemical Society Reviews 38, 2887-2921 (2009).
57. A. Weiss, J. D. Stobo, Requirement for the coexpression of t3 and the T cell antigen receptor on a malignant human T cell line. J. Exp. Med. 160, 1284-1299 (1984).
58. F. W. Studier, Protein production by auto-induction in high density shaking cultures. Protein Expr Purif 41, 207-234 (2005).
59. Hughes, C. E., J. M. Auger, J. McGlade, J. A. Eble, A. C. Pearce, and S. P. Watson, Differential roles for the adapters Gads and LAT in platelet activation by GPVI and CLEC-2. Journal of Thrombosis and Haemostasis 6:2152-2159. (2008).

1. An agent which inhibits Gads (SEQ ID NO: 1) dimerization, said agent interacting with a pharmacophore binding site comprising an amino acid selected from the group consisting of F55, P56, W58, F59, E61, G62, A84-F92, V107-N111, Y115, F116, L125 and N126 of SEQ ID NO: 1.

2. An agent which inhibits Gads (SEQ ID NO: 1) dimerization, said agent interacting with a pharmacophore binding site comprising an amino acid sequence of an SH3 domain of SEQ ID NO: 1.

3. A pharmaceutical composition comprising, as an active ingredient, the agent of claim 1 and a pharmaceutically acceptable carrier or excipient.

4. A method of inhibiting activation of a T cell and/or a mast cell, the method comprising contacting the T cell and/or the mast cell with the agent of claim 1, thereby inhibiting activation of the T cell and/or the mast cell.

5. The method of claim 4, wherein said mast cell activation is FcεRI dependent.

6. (canceled)

7. The method of claim 4, wherein said T cell activation is TCR dependent.

8. The method of claim 4, wherein said T cell is an effector T cell.

9. The method of claim 4, wherein said T cell is a regulatory T cell.

10. (canceled)

11. A method of treating or preventing an allergic response in a subject in need thereof, the method comprising administering to the subject a therapeutically effective amount of the agent of claim 1, thereby treating or preventing the allergic response in the subject.

12. (canceled)

13. A method of treating or preventing a disease associated with activation of T cells in a subject in need thereof, the method comprising administering to the subject a therapeutically effective amount of the agent of claim 1, thereby treating or preventing the disease associated with activation of T cells in the subject.

14. (canceled)

15. The method of claim 13, wherein said T cells are effector T cells.

16. The method of claim 15, wherein said disease is an autoimmune disease.

17. The method of claim 13, wherein said T cells are regulatory T cells.

18. The method of claim 17, wherein said disease is chronic inflammation or cancer.

19-20. (canceled)

21. The agent of claim 1, wherein said agent is a peptide.

22. The agent of claim 21, wherein said peptide comprises an amino acid sequence selected from the group consisting of PGDF (SEQ ID NO: 33), MRDT (SEQ ID NO: 34), MRDN (SEQ ID NO: 38), PGDFGVMRD (SEQ ID NO: 39), PGDFGGVMRD (SEQ ID NO: 40), PGDFPVMRD (SEQ ID NO: 41), ASQSSPGDF (SEQ ID NO: 35), VMRDT (SEQ ID NO: 36), VMRDN (SEQ ID NO: 42) and ASQSSPGDFGVMRD (SEQ ID NO: 43).

23. (canceled)

24. The agent of claim 1, wherein said agent is a small molecule or an antibody.

25. (canceled)

26. The agent of claim 1, wherein said amino acid is selected from the group consisting of A84-F92 and V107-N111.

27. The agent of claim 2, wherein said SH3 domain is located N-terminally to a SH2 domain of said SEQ ID NO: 1.

28. The agent of claim 2, wherein said SH3 domain comprises an amino acid sequence of SEQ ID NO: 50.
